(19) United States
(12) Patent Application Publication
(10) Pub. No.: US 2025/0345345 A1
(43) Pub. Date: Nov. 13, 2025

(54) METHODS FOR THE TREATMENT OF NEUROLOGICAL DISORDERS

(71) Applicant: Praxis Precision Medicines, Inc., Boston, MA (US)

(72) Inventors: STEVEN PETROU, Eltham, Victoria (AU); BERNARD RAVINA, Newton, MA (US); Marion WITTMANN, Medford, MA (US)

(73) Assignee: Praxis Precision Medicines, Inc., Boston, MA (US)

(21) Appl. No.: 18/859,867

(22) PCT Filed: Apr. 24, 2023
(86) PCT No.: PCT/US2023/019656

§ 371 Date: Oct. 24, 2024

Related U.S. Application Data
(60) Provisional application No. 63/334,876, filed on Apr. 26, 2022.

(51) Int. Cl. A61K 31/58; A61K 31/437; A61K 31/4985; A61P 25/08

(52) U.S. Cl. CPC A61K 31/58; A61K 31/437; A61K 31/4985; A61P 25/08

(57) ABSTRACT

The disclosure relates to methods of treating a neurological disorder, such as epilepsy or an epilepsy syndrome, with Compound 1 or pharmaceutically acceptable salts thereof, alone, or in combination with sodium ion (Na⁺) channel blockers.

### CROSS-REFERENCE TO RELATED APPLICATION

This application claims priority to U.S. Provisional Patent Application No. 63/334,876, filed Apr. 26, 2022, which is incorporated herein by reference in its entirety.

### FIELD OF THE DISCLOSURE

The present disclosure relates to methods for the treatment of neurological disorders, such as epilepsy or an epilepsy syndrome, using neurosteroids, such as 3α-hydroxy-3β-methoxymethyl-21-(1′-imidazolyl)-5α-pregnan-20-one, and pharmaceutically acceptable salts thereof, alone, or in combination with sodium ion (Na⁺) channel blockers.

### BACKGROUND OF THE DISCLOSURE

3α-Hydroxy-3β-methoxymethyl-21-(1′-imidazolyl)-5α-pregnan-20-one (Compound 1) is a synthetic neuroactive steroid. Its primary molecular target is the γ-aminobutyric acid type A (GABA_{A}) receptor, where it acts as a positive allosteric modulator (PAM) of channel function. The structural formula of Compound 1 appears below.

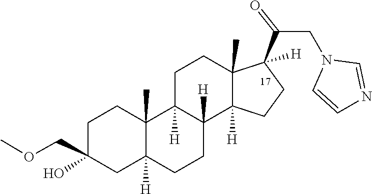

Neuroactive steroid GABA_{A} PAMs have demonstrated clinical efficacy in anesthesia, epilepsy, post-partum depression, and major depression. Genetic variation in the GABA_{A} receptor 8 subunit has been implicated in affecting GABA current amplitude resulting in a susceptibility in humans to epilepsy. Dibbens, L. M., GABRD encoding a protein for extra-or peri-synaptic GABA_{A} receptors is a susceptibility locus for generalized epilepsies, Hum Mol Genet. 2004, 13 (13): 1315-19.

Na_{V} channels are also an important therapeutic target for antiepileptic drugs (AEDs). Their blockade, and consequent inhibition of neuronal sodium current (I_{Na}), is ideally positioned to reduce excitability, as peak I_{Na} in the axonal initial segment and node of Ranvier is responsible for the initiation and propagation of action potentials (APs), respectively. However, the clinical utility of standard Na_{V}-targeting AEDs is limited because current agents, including carbamazepine (CBZ), oxcarbazepine, and phenytoin, can show severe toxicity at therapeutic doses. This toxicity includes ataxia, lethargy, vomiting, and seizures and reflects compromised physiologic neuronal function as a result of excessive peak I_{Na} inhibition or off-target (non-Na_{V}-mediated) activities. Identification of Na channel blockers with improved tolerability or the ability to reduce the dosage of Na channel blockers through, for example, combination therapy, would thus represent a clinically meaningful alternative treatment option.

Around 50 million people globally are believed to be living with epilepsy. Despite the prevalence and negative impact of the disorder, there are few available pharmacotherapies, and their efficacy has substantial limitations. Accordingly, there is an unmet medical need for novel therapeutic compounds for the treatment of neurological disorders such as epilepsy or an epilepsy syndrome and its associated symptoms.

### SUMMARY OF THE DISCLOSURE

The present disclosure, among other things, provides methods of treating a neurological disorder by administering a therapeutically effective amount of a Compound 1 or a pharmaceutically acceptable salt thereof to a patient in need thereof. In some embodiments, the neurological disorder is epilepsy or an epilepsy syndrome. In some embodiments, the method comprises orally administering a daily dose of about 5 mg to about 120 mg, such as about 20 mg, about 40 mg, or about 60 mg, of Compound 1 or a pharmaceutically acceptable salt thereof to a patient in need thereof.

In some embodiments, the neurological disorder is early-onset developmental and epileptic encephalopathy (DEE). In certain embodiments, the neurological disorder is DEE, including, for example, Ohtahara Syndrome; epilepsy with migrating focal seizures of infancy (EIMFS); infantile and childhood DEE, for example West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy. In certain embodiments, the neurological disorder is late seizure onset epileptic encephalopathy. In certain embodiments, the neurological disorder is Benign Familial Neonatal-Infantile Seizures. In certain embodiments, the neurological disorder is an intellectual disability (ID). In certain embodiments, the neurological disorder is an autism spectrum disorder (ASD).

Also provided are methods useful for ameliorating at least one symptom or hallmark of a neurological disorder, such as epilepsy or epilepsy syndrome, including, for example, early-onset DEE, in a subject in need thereof. In certain embodiments, the symptom or hallmark includes one or more of seizures, hypotonia, sensory issues, such as sensory integration disorders, motor dysfunctions, intellectual and cognitive dysfunctions, movement and balance dysfunctions, such as choreoathetosis, dystonia, and ataxia, anxiety, sensory issues, urinary retention problems, irritability, behavior issues, visual dysfunctions, delayed language and speech, gastrointestinal disorders (for example, gastroesophageal reflux, diarrhea, constipation, dysmotility, and the like), neurodevelopmental delays, sleep problems, sudden unexpected death in epilepsy, motor development delays, delayed social milestones, repetitive actions, uncoordinated oral movements. In certain embodiments, the seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures).

In some embodiments, the administration of Compound 1 or a pharmaceutically acceptable salt thereof is in the evening, such as about two hours after an evening meal or about four hours after an evening meal. In certain embodiments, the administration of Compound 1 or a pharmaceutically acceptable salt thereof is between about 5 p.m. and about 12 a.m., and in certain embodiments, the administration of Compound 1 or a pharmaceutically acceptable salt thereof is within about two hours prior to bedtime or at about bedtime. In some embodiments, the Compound 1 or a pharmaceutically acceptable salt thereof is administered for about 1 week to about 4 weeks.

In certain embodiments of the methods disclosed herein, after administering the Compound 1 or a pharmaceutically acceptable salt thereof, the patient experiences a reduction of seizures compared to prior to the treatment. In certain embodiments, the reduction in seizures is a reduction in clonic seizures, and in certain embodiments, the reduction in seizures is a reduction in tonic seizures. In certain embodiments, the reduction is seizures is a reduction is clonic and tonic seizures.

In certain embodiments of the methods disclosed herein, administering Compound 1 or a pharmaceutically acceptable salt thereof provides a mean steady state AUC₀₋₂₄ of from about 600 ng·h/mL to about 900 ng·h/mL, and in certain embodiments, administering Compound 1 or a pharmaceutically acceptable salt thereof provides a mean steady state Cmax of from about 125 ng/ml to about 250 ng/mL.

In certain embodiments, the methods disclosed herein further comprise administering at least one antiepileptic agent, such as brivaracetam, cannabidiol, carbamazepine, clobazam, clonazepam, diazepam, divalproex, eslicarbazepine, ethosuximide, ezogabine, fenfluramine, felbamate, gabapentin, lacosamide, lamotrigine, levetiracetam, lorazepam, neuroactive steroids, oxcarbezepine, permpanel, phenobarbital, phenytoin, pregabalin, primidone, rufinamide, tigabine, topiramate, valproic acid, vigabatrin, and zonisamide, to the patient. In certain embodiments disclosed herein, the Compound 1 is a salt, such as a citrate salt, including, for example, a hemi-citrate salt.

In certain embodiments, the methods disclosed herein further comprise administering at least one sodium channel blocker to the patient, such as a sodium channel blocker of Compound A, or a pharmaceutically acceptable salt thereof, or a sodium channel blocker of Compound B, or a pharmaceutically acceptable salt thereof. In certain embodiments of the methods disclosed herein, about 0.1 mg to about 100 mg of the at least one sodium channel blocker is administered per day.

Also disclosed herein is a Compound 1 or pharmaceutically acceptable salt thereof for use in treating a neurological disorder, such as epilepsy or an epilepsy syndrome, alone or in combination with at least one sodium channel blocker to the patient, such as a sodium channel blocker of Compound A or Compound B, or a pharmaceutically acceptable salt thereof.

### BRIEF DESCRIPTION OF THE FIGURES

FIG. 1 is a graph showing the number of phenylenetetrazole (“PTZ”)-induced tonic seizures recorded for mice after administration of vehicle controls, valproate (VPA), or Compound 1 (at 3, 10, or 30 mg/kg), as described in Example 1.

FIG. 2 is a graph showing the number of PTZ-induced clonic seizures recorded for mice after administration of vehicle controls, valproate (VPA), or Compound 1 (at 3, 10, or 30 mg/kg), as described in Example 1.

FIG. 3 is a graph showing the number of PTZ-induced clonic seizures recorded for mice after administration of a vehicle control, Compound A (1 mg/kg), Compound 1 (10 mg/kg) or a combination of Compound A (1 mg/kg) and Compound 1 (10 mg/kg), as described in Example 2.

### DETAILED DESCRIPTION

### Definitions

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms “disease,” “disorder,” and “condition” are used interchangeably herein.

As used herein, the term “in some embodiments,” “in other embodiments,” or the like, refers to embodiments of all aspects of the disclosure, unless the context clearly indicates otherwise.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of Compound 1 or a pharmaceutically acceptable salt thereof is that amount that is required to reduce at least one symptom of epilepsy in a patient. The actual amount that comprises the “effective amount” or “therapeutically effective amount” will vary depending on a number of conditions including, but not limited to, the severity of the disorder, the size and health of the patient, and the route of administration.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “treating” as used herein with regard to a patient, refers to improving at least one symptom of the patient's disorder. Treating can be improving, or at least partially ameliorating a disorder. The term “therapeutic effect” as used herein refers to a desired or beneficial effect provided by the method and/or the composition. For example, the method for treating epilepsy provides a therapeutic effect when the method reduces at least one symptom of epilepsy in a patient.

The phrase “in combination with” as used herein refers to administration to a subject in need thereof of a compound, such as Compound 1 or a pharmaceutically acceptable salt thereof, and at least one sodium channel ion blocker or a pharmaceutically acceptable salt thereof, such as Compound A or Compound B, whereby the subject has an active prescription for Compound 1 or a pharmaceutically acceptable salt thereof and an active prescription for at least one sodium channel ion blocker or a pharmaceutically acceptable salt thereof, and is being directed by a physician to take Compound 1 or a pharmaceutically acceptable salt thereof and at least one sodium channel ion blocker or a pharmaceutically acceptable salt thereof. In some embodiments, the term “in combination” also refers to administration to a subject in need thereof of Compound 1 or a pharmaceutically acceptable salt thereof and of at least one sodium channel ion blocker or pharmaceutically acceptable salt thereof over the same period of time. In some embodiments, the Compound 1 or a pharmaceutically acceptable salt thereof and the at least one sodium channel ion blocker or a pharmaceutically acceptable salt thereof may be administered in combination to a subject in need thereof each according to the same administration schedule or each according to different administration schedules. For example, in some embodiments, Compound 1 or a pharmaceutically acceptable salt thereof and at least one sodium channel ion blocker or a pharmaceutically acceptable salt thereof may each be administered to a subject in need thereof over the same period of time once daily, e.g., in the morning. In other embodiments, Compound 1 or a pharmaceutically acceptable salt thereof may be administered to a subject once daily, e.g., in the morning, and at least one sodium channel ion blocker or a pharmaceutically acceptable salt thereof may be administered to a subject in need thereof two or three times daily over the same period of time. In some embodiments, Compound 1 or a pharmaceutically acceptable salt thereof and at least one sodium channel ion blocker or a pharmaceutically acceptable salt thereof may be administered to a subject in need thereof simultaneously as a part of a new pharmaceutical composition. In other embodiments, Compound 1 or a pharmaceutically acceptable salt thereof and at least one sodium channel ion blocker or a pharmaceutically acceptable salt thereof may be administered in combination simultaneously, or within several hours or minutes, to a subject in need thereof as parts of different pharmaceutical compositions.

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described, for example, in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C_{2- 8} alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

“Hetero” when used to describe a compound or a group present on a compound means that one or more carbon atoms in the compound or group have been replaced by a nitrogen, oxygen, or sulfur heteroatom. Hetero may be applied to any of the alkyl groups described above such as alkyl, e.g., heteroalkyl; alkenyl, e.g., heteroalkenyl; alkynyl, e.g., heteroalkynyl; carbocyclyl, e.g., heterocyclyl; aryl, e.g., heteroaryl, and the like having from 1 to 5, and particularly from 1 to 3 heteroatoms.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₅ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (Ca), cyclooctenyl (Ca), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro|4.5|decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spire ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Cyano” refers to —CN.

“Halo” or “halogen” refers to a fluorine atom (i.e., fluoro or —F), a chlorine atom (i.e., chloro or —Cl), a bromine atom (i.e., bromo or —Br), and an iodine atom (i.e., iodo or —I). In certain embodiments, the halo group is fluoro or chloro.

“Haloalkyl” refers to an alkyl group substituted with one or more halogen atoms.

“Nitro” refers to —NO₂.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

The embodiments disclosed herein are not intended to be limited in any manner by the above exemplary listing of chemical groups and substituents. Those skilled in the art will recognize that several embodiments are possible within the scope and spirit of the present disclosure. The following description illustrates the disclosure and, of course, should not be construed in any way as limiting the scope of the inventions described herein

### Neurological Disorders

Disclosed herein are methods of treating a neurological disorder by administering a therapeutically effective amount of a neuroactive steroid such as Compound 1 or a pharmaceutically acceptable salt thereof to a patient in need thereof. Also disclosed are methods of treating a neurological disorder by administering a therapeutically effective amount of a neuroactive steroid such as Compound 1 or a pharmaceutically acceptable salt thereof in combination with a therapeutically effective amount of a sodium channel blocker, such as Compound A or Compound B, or a pharmaceutically acceptable salt thereof.

In certain embodiments, the compounds described herein are useful in the treatment of epilepsy and epilepsy syndromes. Epilepsy is a CNS disorder in which nerve cell activity in the brain becomes disrupted, causing seizures or periods of unusual behavior, sensations and sometimes loss of consciousness. Seizure symptoms will vary widely, from a simple blank stare for a few seconds to repeated twitching of their arms or legs during a seizure.

Epilepsy may involve a generalized seizure or a partial or focal seizure. All areas of the brain are involved in a generalized seizure. A person experiencing a generalized seizure may cry out or make some sound, stiffen for several seconds to a minute a then have rhythmic movements of the arms and legs. The eyes are generally open, the person may appear not to be breathing and actually turn blue. The return to consciousness is gradual and the person maybe confused from minutes to hours. There are six main types of generalized seizures: tonic-clonic, tonic, clonic, myoclonic, absence, and atonic seizures. In a partial or focal seizure, only part of the brain is involved, so only part of the body is affected. Depending on the part of the brain having abnormal electrical activity, symptoms may vary.

Epilepsy, as described herein, includes a generalized, partial, complex partial, tonic clonic, clonic, tonic, refractory seizures, status epilepticus, absence seizures, febrile seizures, or temporal lobe epilepsy.

The compounds described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that may be resistant to treatment and cause severe cognitive dysfunction, for instance West syndrome.

In some embodiments, the epilepsy syndrome comprises an epileptic encephalopathy, such as Dravet syndrome, Angelman syndrome, CDKL5 disorder, frontal lobe epilepsy, infantile spasms, West syndrome, Juvenile Myoclonic Epilepsy, Landau-Kleffner syndrome, Lennox-Gastaut syndrome, Ohtahara syndrome, PCDH19 epilepsy, or Glut1 deficiency.

In some embodiments, the epilepsy or epilepsy syndrome is a genetic epilepsy or a genetic epilepsy syndrome. In some embodiments, the epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy (SUDEP), Rasmussen encephalitis, malignant migrating partial seizures of infancy, autosomal dominant nocturnal frontal lobe epilepsy, KCNQ2 epileptic encephalopathy, or KCNT1 epileptic encephalopathy.

In some embodiments, the neurological disorder is a disorder associated with excessive neuronal excitability or a disorder associated with a gain-of-function mutation in a gene (e.g., KCNT1), such as, for example, malignant migrating focal seizures of infancy (MMFSI) or epilepsy of infancy with migrating focal seizures (EIMFS), autosomal dominant nocturnal frontal lobe epilepsy (ADNFLE), or West syndrome. In some embodiments, the neurological disorder is selected from infantile spasms, epileptic encephalopathy, focal epilepsy, Ohtahara syndrome, developmental and epileptic encephalopathy (DEE), or Lennox-Gastaut syndrome. In some embodiments, the neurological disorder is seizure. In some embodiments, the neurological disorder is selected from cardiac arrhythmia, Brugada syndrome, or myocardial infarction.

In some embodiments, the neurological disorder is developmental and epileptic encephalopathy (DEE). DEEs include a broad range of diseases that include neonatal and early infantile DEE, for example Ohtahara Syndrome and epilepsy with migrating focal seizures of infancy (EIMFS); infantile and childhood DEE, for example West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy (Wolff, M., et al., 2019; Harkin, L. A., et al., 2007, Brain 130, 843-852; Escayg, A., et al., 2010, Epilepsia 51, 1650-1658; Miller I. O, et al., 2007 Nov. 29 [Updated 2019 Apr. 18]. In: Adam M P, Ardinger H H, Pagon R A, et al., editors. GeneReviews® [Internet]. Seattle (WA): University of Washington, Seattle; 1993-2020.

In some embodiments, the DEE is early-onset DEE. Early-onset DEE is caused by gain-of-function (GoF) variants in the SCN2A gene encoding the voltage-gated sodium channel Nav1.2. This is a rare, severe, and life-threatening condition characterized by drug-resistant epilepsy which is widely reported to have onset of seizures in the first three months of life (Wolff et al 2017, Wolff et al 2019) Error! Reference source not found. In addition to frequent seizures, patients with early-onset DEE have a profound developmental impairment and extensive comorbidities including limitations in communication with most children being non-verbal; autonomic dysfunction; developmental delay; GI abnormalities; movement disorders, such as choreoathetosis, dystonia, ataxia; anxiety; sensory issues; urinary retention problems; and diminished quality of life resulting from (and characterized by) severe irritability, variable sleep problems (e.g., inability to fall and stay asleep), behavior issues, frequent ER visits and hospitalizations, and total reliance on caregivers.

Also provided are methods useful for ameliorating at least one symptom or hallmark of a neurological disorder, such as epilepsy or epilepsy syndrome, including, for example, early-onset DEE, in a subject in need thereof. In certain embodiments, the symptom or hallmark includes one or more of seizures, hypotonia, sensory issues, such as sensory integration disorders, motor dysfunctions, intellectual and cognitive dysfunctions, movement and balance dysfunctions, such as choreoathetosis, dystonia, and ataxia, anxiety, sensory issues, urinary retention problems, irritability, behavior issues, visual dysfunctions, delayed language and speech, gastrointestinal disorders (for example, gastroesophageal reflux, diarrhea, constipation, dysmotility, and the like), neurodevelopmental delays, sleep problems, sudden unexpected death in epilepsy, motor development delays, delayed social milestones, repetitive actions, uncoordinated oral movements. In certain embodiments, the seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures).

In some embodiments, the neurological disorder is selected from a learning disorder, Fragile X, intellectual function, neuronal plasticity, a psychiatric disorder, or an autism spectrum disorder.

EIMFS is a rare and debilitating genetic condition characterized by an early onset (before 6 months of age) of almost continuous heterogeneous focal seizures, where seizures appear to migrate from one brain region and hemisphere to another. Patients with EIMFS are generally intellectually impaired, non-verbal and non-ambulatory. While several genes have been implicated to date, the gene that is most commonly associated with EIMFS is KCNT1. Several de novo mutations in KCNT1 have been identified in patients with EIMFS, including V271F, G288S, R428Q, R474Q, R474H, R474C, I760M, A934T, P924L, G243S, H257D, A259D, R262Q, Q270E, L274I, F346L, C377S, R398Q, P409S, A477T, F502V, M516V, Q550del, K629E, K629N, I760F, E893K, M896K, R933G, R950Q, and K1154Q. Barcia et al. (2012) Nat Genet. 44:1255-1260; Ishii et al. (2013) Gene 531:467-471; McTague et al. (2013) Brain. 136:1578-1591; Epi4K Consortium & Epilepsy Phenome/Genome Project. (2013) Nature 501:217-221; Lim et al. (2016) Neurogenetics; Ohba et al. (2015) Epilepsia 56: el21-el28; Zhou et al. (2018) Genes Brain Behav. e12456; Moller et al. (2015) Epilepsia. e114-20; Numis et al. (2018) Epilepsia. 1889-1898; Madaan et al. Brain Dev. 40 (3): 229-232; McTague et al. (2018) Neurology. 90 (1): e55-e66; Kawasaki et al. (2017) J Pediatr. 191:270-274; Kim et al. (2014) Cell Rep. 9 (5): 1661-1672; Ohba et al. (2015) Epilepsia. 56 (9): e121-8; Rizzo et al. (2016) Mol Cell Neurosci. 72:54-63; Zhang et al. (2017) Clin Genet. 91 (5): 717-724; Mikati et al. (2015) Ann Neurol. 78 (6): 995-9; Baumer et al. (2017) Neurology. 89 (21): 2212; Dilena et al. (2018) Neurotherapeutics. 15 (4): 1112-1126. These mutations may be gain-of-function, missense mutations that are dominant (i.e., present on only one allele) and result in change-in-function of the encoded potassium channel that causes a marked increase in whole cell current when tested in Xenopus oocyte or mammalian expression systems (see e.g. Milligan et al. (2015) Ann Neurol. 75 (4): 581-590; Barcia et al. (2012) Nat Genet. 44 (11): 1255-1259; and Mikati et al. (2015) Ann Neurol. 78 (6): 995-999).

ADNFLE has a later onset than EIMFS, generally in mid-childhood, and is generally a less severe condition. It is characterized by nocturnal frontal lobe seizures and can result in psychiatric, behavioral and cognitive disabilities in patients with the condition. While ADNFLE is associated with genes encoding several neuronal nicotinic acetylcholine receptor subunits, mutations in the KCNT1 gene have been implicated in more severe cases of the disease (Heron et al. (2012) Nat Genet. 44:1188-1190). Functional studies of the mutated KCNT1 genes associated with ADNFLE indicated that the underlying mutations (M8961, R398Q, Y796H, and R928C) were dominant, gain-of-function mutations (Milligan et al. (2015) Ann Neurol. 75 (4): 581-590; Mikati et al. (2015) Ann Neurol. 78 (6): 995-999).

West syndrome is a severe form of epilepsy composed of a triad of infantile spasms, an interictal electroencephalogram (EEG) pattern termed hypsarrhythmia, and mental retardation, although a diagnosis can be made one of these elements is missing. Mutations in KCNT1, including G652V and R474H, have been associated with West syndrome (Fukuoka et al. (2017) Brain Dev 39:80-83 and Ohba et al. (2015) Epilepsia 56: el21-el28). Treatment targeting the KCNT1 channel suggests that these mutations are gain-of-function mutations (Fukuoka et al. (2017) Brain Dev 39:80-83).

Exemplary diseases, disorders, or conditions include epilepsy and other encephalopathies (e.g., MMFSI or EIMFS, ADNFLE, West syndrome, infantile spasms, epileptic encephalopathy, DEE, early infantile epileptic encephalopathy (EIEE), generalized epilepsy, focal epilepsy, multifocal epilepsy, temporal lobe epilepsy, Ohtahara syndrome, early myoclonic encephalopathy, Lennox-Gastaut syndrome, drug resistant epilepsy, seizures (e.g., frontal lobe seizures, generalized tonic clonic seizures, asymmetric tonic seizures, focal seizures), leukodystrophy, hypomyelinating leukodystrophy, and leukoencephalopathy), cardiac dysfunctions (e.g., cardiac arrhythmia, Brugada syndrome, myocardial infarction), pulmonary vasculopathy/hemorrhage, pain and related conditions (e.g., neuropathic pain, acute/chronic pain, migraine, etc.), muscle disorders (e.g., myotonia, neuromyotonia, cramp muscle spasms, spasticity), itch and pruritis, movement disorders (e.g., ataxia and cerebellar ataxias), psychiatric disorders (e.g., major depression, anxiety, bipolar disorder, schizophrenia, attention-deficit hyperactivity disorder), neurodevelopmental disorder, learning disorders, intellectual disability, Fragile X, neuronal plasticity, and autism spectrum disorders.

In certain embodiments, the compounds disclosed herein (e.g., Compound 1 or a pharmaceutically acceptable salt thereof) may also be used to treat an epileptic encephalopathy, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCNIA, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

In some embodiments, the methods described herein further comprise identifying a subject having a neurological disorder, such as epilepsy or an epilepsy syndrome, prior to administration of a compound described herein (e.g., Compound 1 or a pharmaceutically acceptable salt thereof).

In some embodiments, the present methods employ a neuroactive steroid, such as Compound 1 as described herein, or a pharmaceutically acceptable salt thereof, alone or in combination with other therapeutic agents, such as a sodium channel blocker.

### Compound 1

3α-Hydroxy-3β-methoxymethyl-21-(1′-imidazolyl)-5α-pregnan-20-one (Compound 1) is a synthetic neuroactive steroid. The structural formula of Compound 1 appears below.

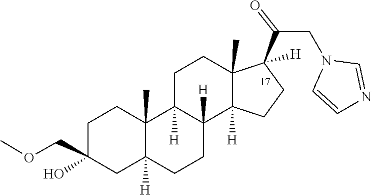

Compound 1 is a GABA_{A} positive allosteric modulator (PAM) with high potency similar to clinical stage neuroactive steroids (allopregnanolone, ganaxolone, SAGE-217, alphaxolone). The synthesis of Compound 1 is described in U.S. Publication Nos. 2004/034002 and 2009/0118248; crystalline polymorph of Compound 1 free base is described in U.S. Publication No. 2006/0074059; pharmaceutical compositions containing Compound 1 are described in U.S. Publication No. 2009/0131383, which are hereby incorporated by reference in their entirety for all purposes.

Compound 1 acts as a PAM of both extrasynaptic and synaptic GABA_{A} receptors, in contrast to the benzodiazepines that primarily impact synaptic GABA_{A} receptors. Compound 1 shows a greater preference for extrasynaptic over synaptic human GABA_{A} receptors than other NAS GABA PAMs, demonstrating about a 6-fold preference in terms of potency, and about a 10.5-fold preference in terms of efficacy. Compound 1 preferentially potentiates the delta subunit of the GABA_{A} receptor, which sits in the extrasynaptic space. This preference for extrasynaptic over synaptic human GABA_{A} receptors is hypothesized to result in less somnolence/sedation at potentially therapeutic exposures than other neuroactive steroids, including other neuroactive steroids having a greater proportional impact on synaptic receptors.

Accordingly, in one aspect, the present disclosure provides a method of treating a neurological disorder, such as epilepsy or an epilepsy syndrome comprising administering an effective amount of a neuroactive steroid, such as Compound 1 or a pharmaceutically acceptable salt thereof, to a patient in need of such treatment. In accordance with some embodiments of the present disclosure, at least about 5 mg, about 10 mg, about 15 mg, about 30 mg, about 45 mg, about 60 mg, about 75 mg, about 80 mg, about 85 mg, about 90 mg, about 95 mg, about 100 mg, about 105 mg, about 110 mg, about 115 mg or about 120 mg of Compound 1 or a pharmaceutically acceptable salt thereof is administered. In accordance with some embodiments of the present disclosure, no more than about 5 mg, about 10 mg, about 15 mg, about 30 mg, about 45 mg, about 60 mg, about 75 mg, about 80 mg, about 85 mg, about 90 mg, about 95 mg, about 100 mg, about 105 mg, about 110 mg, about 115 mg or about 120 mg of Compound 1 or a pharmaceutically acceptable salt thereof is administered.

In another aspect, the present disclosure provides methods of administering a neuroactive steroid, such as Compound 1 or a pharmaceutically acceptable salt thereof, that reduce the adverse events (AEs) that may be associated with administration of other GABA_{A} PAMs (e.g., somnolence, sedation, dizziness, lightheadedness, fatigue, euphoria, feeling drunk, feeling abnormal, headache, cognitive disorder, disturbance of attention, memory impairment, slowed cognition, decreased alertness, constipation, and diarrhea). In some embodiments, the methods of the present disclosure reduce the incidence of AEs. In some embodiments, the methods of the present disclosure reduce the severity of AEs. As used herein, the incidence and severity of an AE is determined according to guidelines set forth in Guidance for Industry and Investigators: Safety Reporting Requirements for INDs and BA/BE Studies (2012), described in the “NIH-FDA Phase 2 and 3 IND/IDE Clinical Trial Protocol Template”, and reported with standardized terms according to the Medical Dictionary for Regulatory Activities (MedDRA).

In certain embodiments of the disclosure, the administration of Compound 1 in the evening provides pharmacokinetic parameters that are associated with reduced AEs without decreasing the therapeutic effectiveness of Compound 1, as disclosed, for example, in PCT Publication No. WO 2022/232104, incorporated by reference herein. Specifically, evening administration of Compound 1 reduces the maximum observed plasma concentration (Cₘₐₓ) and increases the time to maximum plasma concentration (tₘₐₓ) compared to morning administration of Compound 1 while providing a similar overall drug exposure (as determined by AUC_{inf}). The fact that evening administration of Compound 1 does not change the AUC_{inf} enables a patient to receive a similar total drug exposure over time while maintaining a lower Cₘₐₓ, which reduces AEs.

It is difficult to predict how the time of dosing impacts a particular drug's pharmacokinetic (PK) behaviors because multiple factors that impact drug PK are related to circadian variations, including protein binding, absorption from the gastrointestinal tract, the blood flow fraction an organ receives, and liver enzyme activity, as well as the physicochemical properties of the particular drug.

The difficulty in predicting the effect of dosing time on the pharmacokinetics and therapeutic effect of a therapeutic is exemplified by amlodipine besylate. Like Compound 1, amlodipine is lipophilic. Unlike Compound 1, evening administration of amlodipine besylate is associated with a reduced Tₘₐₓ and increased Cmax compared to morning dosing. Khodadoustan, et al., Clinical and Experimental Hypertension 2017; 39 (6): 520-526. In contrast, and unexpectedly, evening dosing of Compound 1 provides an increase in Tₘₐₓ and decrease in Cₘₐₓ, which is associated with reduced side effects. Thus, patients treated with Compound 1 according to the methods described herein experience a therapeutic benefit with reduced adverse events.

In some embodiments, the Compound 1 used in the compositions and methods of the present disclosure is a pharmaceutically acceptable salt of Compound 1. Salts of Compound 1 and polymorphs thereof are described in U.S. Pat. No. 10,562,930, which is hereby incorporated by reference in its entirety. In some embodiments, the pharmaceutically acceptable salt of Compound 1 used in the compositions and methods of the present disclosure is selected from hydrobromide, citrate, malate, mesylate, phosphate, tartrate, hydrochloride, tosylate, glucuronate, ethanesulfonate, fumarate, sulfate, napthalene-2-sulfonate, ascorbate, oxalate, napthalene-1,5-disulfonate, malonate, aminosalicylate, benzenesulfonate, isethionate, gentistate, 1-hydroxy-2-napthoate, dichloroacetate, cyclamate, and ethane-1,2-disulfonate salts. In certain embodiments, the salt of Compound 1 is Compound 1 hydrobromide. In certain embodiments, the salt of Compound 1 is Compound 1 citrate. In certain embodiments, the salt of Compound 1 is Compound 1 mono-citrate. In certain embodiments, the salt of Compound 1 is Compound 1 hemi-citrate. In certain embodiments, the salt of Compound 1 is Compound 1 L-malate. In certain embodiments, the salt of Compound 1 is Compound 1 mesylate. In certain embodiments, the salt of Compound 1 is Compound 1 phosphate. In certain embodiments, the salt of Compound 1 is Compound 1 L(+)-tartrate. In certain embodiments, the salt of Compound 1 is Compound 1 hydrochloride. In certain embodiments, the salt of Compound 1 is Compound 1 tosylate. In certain embodiments, the salt of Compound 1 is Compound 1 glucuronate. In certain embodiments, the salt of Compound 1 is Compound 1 ethanesulfonate.

### Compositions

Compound 1 as employed in the present methods can form a part of a pharmaceutical composition by combining Compound 1, or a pharmaceutically acceptable salt thereof, with a pharmaceutically acceptable carrier. Additionally, the compositions can include at least one additive selected from adjuvants, excipients, diluents, release-modifying agents, lubricants (e.g., magnesium stearate), and stabilizers. The composition can be an immediate release formulation, a delayed release formulation, a sustained release formulation or an extended release formulation.

The methods of the present disclosure can employ various compositions for administration to patients, e.g., humans. In certain embodiments, the composition comprising Compound 1 may be in unit a dosage form, such as tablets, capsules, pills, powders, granules, sterile parenteral solutions or suspensions (e.g., intramuscular (IM), subcutaneous (SC) and intravenous (IV)), transdermal patches, oral solutions or suspensions, and oil-water emulsions containing suitable quantities of Compound 1 or a pharmaceutically acceptable salt thereof.

Oral pharmaceutical dosage forms can be either solid or liquid. The solid dosage forms can be tablets, capsules, granules, films (e.g., buccal films), and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and suspensions reconstituted from non-effervescent granules and effervescent preparations reconstituted from effervescent granules.

Aqueous solutions include, for example, elixirs and syrups. Emulsions can be either oil-in-water or water-in-oil. Elixirs are clear, sweetened, hydroalcoholic preparations. Pharmaceutically acceptable carriers used in elixirs include solvents. Syrups can be concentrated aqueous solutions of a sugar, such as sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners, and wetting agents. Pharmaceutically acceptable substance used in effervescent granules (for example to be reconstituted into a liquid oral dosage form) can include organic acids and a source of carbon dioxide. Coloring and flavoring agents can be used in all of the above dosage forms.

In some embodiments, the present disclosure provides a pharmaceutical composition comprising a salt of Compound 1. In some embodiments, the salt of Compound 1 is Compound 1 hydrobromide, Compound 1 citrate (such as Compound 1 mono-citrate or Compound 1 hemi-citrate), Compound 1 L-malate, Compound 1 mesylate, Compound 1 phosphate, Compound 1 L(+)-tartrate, Compound 1 hydrochloride, Compound 1 tosylate, Compound 1 glucuronate, or Compound 1 ethanesulfonate.

In some embodiments, the disclosure provides pharmaceutical compositions comprising Compound 1 or a pharmaceutically acceptable salt thereof and a lubricant, such as magnesium stearate, wherein the percentage of the lubricant in the pharmaceutical composition is less than about 4% by weight, such as less than about 3%, less than about 2.5%, or less than about 2% by weight. In some embodiments, the pharmaceutical compositions comprise from about 0.5% to about 3%, such as from about 1% to about 2%, of a lubricant, such as magnesium stearate, by weight (e.g., about 0.5%, about 1%, about 1.5%, about 2%, about 2.5%, or about 3% magnesium stearate by weight). In some embodiments, the pharmaceutical composition further comprises crospovidone, and in certain embodiments, the percentage of crospovidone in the pharmaceutical composition is from about 3% to about 8% by weight. In some embodiments, the disclosure provides pharmaceutical compositions that comprise about 20% to about 30% (e.g., about 20%, about 22%, about 25%, about 27%, about 28%, about 29% or about 30%) by weight of Compound 1 or a pharmaceutically acceptable salt thereof and from about 1% to about 3%, from about 1.5% to about 2.5%, or from about 1.75% to about 2.25% magnesium stearate by weight. In certain embodiments, the pharmaceutical composition comprises about 25% of Compound 1 or a pharmaceutically acceptable salt thereof by weight, about 2% magnesium stearate by weight, and about 7% crospovidone by weight.

### Combination Therapy

While the compositions disclosed herein can be administered as the sole active pharmaceutical ingredient (i.e., Compound 1) in the methods described herein, in some embodiments they can also be used in combination with one or more ingredients which are known to be therapeutically effective against neurological disorders and/or compliment the anti-seizure effect of Compound 1. In some embodiments, Compound 1 or a pharmaceutically acceptable salt thereof is administered in combination with an additional agent by co-formulation as a single composition or by administration as separate compositions.

For example, in some embodiments, the present methods can employ administering Compound 1, or a pharmaceutically acceptable salt thereof, in conjunction with one or more additional anti-epileptic agents. In some embodiments, the present methods can employ administering Compound 1, or a pharmaceutically acceptable salt thereof, in conjunction with one or more GABA_{A} PAM neuroactive steroids. In some embodiments, the present methods can employ administering Compound 1, or a pharmaceutically acceptable salt thereof, in conjunction with one or more approved anti-epileptic agents.

In some embodiments, the present methods can employ administering Compound 1, or a pharmaceutically acceptable salt thereof, in conjunction with one or more sodium channel blockers. In certain embodiments, the sodium channel blocker is a compound of Formula (I):

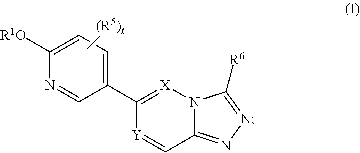

or a pharmaceutically acceptable salt thereof, wherein:
X and Y are each independently CR^{d} or N;
R¹ is

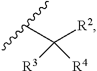

monocyclic C₃₋₆ cycloalkyl, or 4- to 7-membered monocyclic heterocyclyl, wherein said cycloalkyl and heterocyclyl are optionally substituted with one or more R^{a};
R² is C₁₋₄haloalkyl, phenyl, or monocyclic C₃₋₆ cycloalkyl optionally substituted with one or more R^{b};
R³ is hydrogen, C₁₋₄alkyl, or C₁₋₄haloalkyl;
R⁴ is hydrogen or C₁₋₄alkyl;
R⁵ is halo;
R⁶ is C₁₋₄alkyl or C₁₋₄haloalkyl, wherein said C₁₋₄alkyl or C₁₋₄haloalkyl are each substituted with OR^{c};
t is 0, 1, or 2;
R^{a} and R^{b} are each independently selected from is selected from halo, C₁₋₄alkyl, C₁₋₄haloalkyl, C₁₋₄alkoxy, and C₁₋₄haloalkoxy,
R^{c} is C₁₋₄alkyl optionally substituted with C₃₋₆ cycloalkyl or C₁₋₄alkoxy, or C₃₋₆ cycloalkyl; and
R^{d} is hydrogen or C₁₋₄alkyl;
   or a pharmaceutically acceptable salt thereof.

In some In some embodiments, the compound is not a compound having the formula:

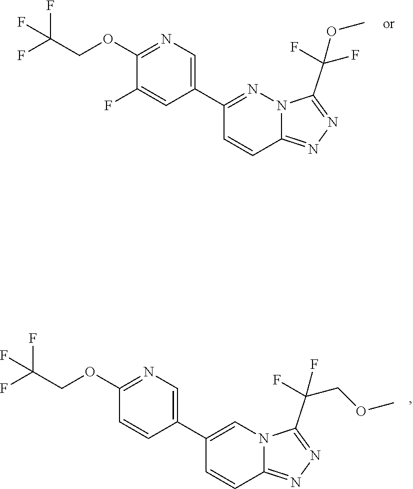 [SMILES: COC(F)(F)c1nnc2ccc(-c3cnc(OCC(F)(F)F)c(F)c3)nn12 ; COCC(F)(F)c1nnc2ccc(-c3ccc(OCC(F)(F)F)nc3)cn12]

or a pharmaceutically acceptable salt thereof.

In some embodiments, the compound is a compound of Formula (I-a):

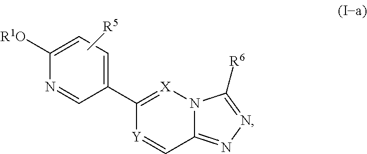

or a pharmaceutically acceptable salt thereof, wherein the variables are as defined herein.

In some embodiments, the compound is a compound of Formula (I-b):

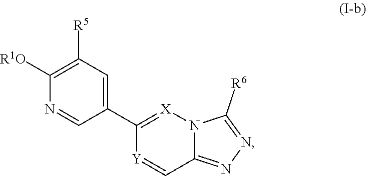

or a pharmaceutically acceptable salt thereof, wherein the variables are as defined herein.

In some embodiments, the compound is a compound of Formula (I-c):

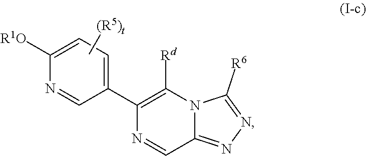

or a pharmaceutically acceptable salt thereof, wherein the variables are as defined herein.

In some embodiments, the compound is a compound of Formula (II):

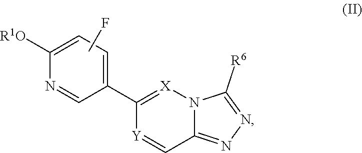

or a pharmaceutically acceptable salt thereof, wherein the variables are as defined herein.

In some embodiments, the compound is a compound of Formula III:

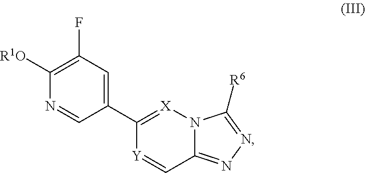

or a pharmaceutically acceptable salt thereof, wherein the variables are as defined herein.

In some embodiments, the compound is a compound of Formula IV:

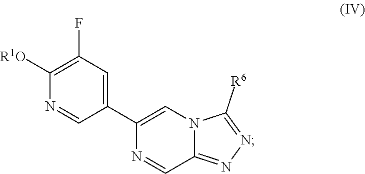

or a pharmaceutically acceptable salt thereof, wherein the variables are as defined herein.

In some embodiments, the present methods can employ administering Compound 1, or a pharmaceutically acceptable salt thereof, in combination with Compound A having the following formula:

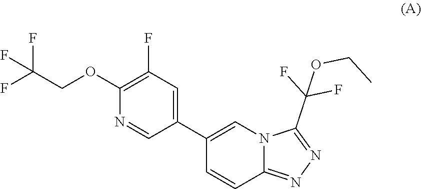 [SMILES: CCOC(F)(F)c1nnc2ccc(-c3cnc(OCC(F)(F)F)c(F)c3)cn12]

or a pharmaceutically acceptable salt thereof.

In certain embodiments, the present methods can employ administering Compound 1, or a pharmaceutically acceptable salt thereof, in combination with Compound B having the following formula:

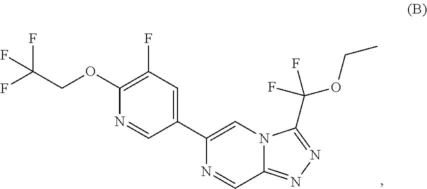 [SMILES: CCOC(F)(F)c1nnc2cnc(-c3cnc(OCC(F)(F)F)c(F)c3)cn12]

or a pharmaceutically acceptable salt thereof.

In certain embodiments, a subject to be administered a compound disclosed herein may have a disease, disorder, or condition, or a symptom thereof, that would benefit from treatment with another agent or therapy. These diseases or conditions can relate to epilepsy or an epilepsy syndrome, a neurodevelopmental disorder, pain, migraine, headache, a neuromuscular disorder, or a psychiatric disorder (e.g., bipolar disorder).

The sodium channel blockers described herein are generally useful for modulating the activity of sodium channels and are useful in treating conditions relating to aberrant function of a sodium channel ion channel, e.g., abnormal late sodium (I_{Na}L) current. In some embodiments, a sodium channel blocker is effective in the treatment of epilepsy or an epilepsy syndrome. Sodium channel blockers, pharmaceutically acceptable salts thereof, or compositions comprising the same may also modulate all sodium ion channels, or may be specific to only one or a plurality of sodium ion channels, e.g., Nav1.1, 1.2, 1.3, 1.4, 1.5, 1.6, 1.7, 1.8, and/or 1.9. In some embodiments, a sodium channel blocker has specificity to sodium ion channel Nav1.6.

In one aspect, provided herein is a dosage form or a composition in a dosage form comprising: from about 0.1 mg to about 500 mg (e.g., from about 0.5 mg to about 200 mg, from about 1 mg to about 150 mg, from about 10 mg to about 120 mg) of a compound of Formula (I), (I-a), (I-b), (I-c), (II), (III), or (IV), Compound A, or Compound B; and a pharmaceutically acceptable excipient.

In some embodiments, the dosage form or a composition in a dosage form of the sodium channel blocker, such as Formula I, (I-a), (I-b), (I-c), (II), (III), or (IV), Compound A, or Compound B, comprises from about 2.5 mg to about 150 mg (e.g., from about 10 mg to about 150 mg, from about 20 mg to about 150 mg, from about 40 mg to about 150 mg, from about 60 mg to about 150 mg, from about 80 mg to about 150 mg, from about 100 mg to about 150 mg, from about 10 mg to about 120 mg, from about 20 mg to about 120 mg, from about 40 mg to about 120 mg, from about 60 mg to about 120 mg, from about 80 mg to about 120 mg, from about 100 mg to about 120 mg, from about 10 mg to about 100 mg, from about 20 mg to about 100 mg, from about 40 mg to about 100 mg, from about 60 mg to about 100 mg, from about 80 mg to about 100 mg, from about 10 mg to about 80 mg, from about 20 mg to about 80 mg, from about 40 mg to about 80 mg, from about 60 mg to about 80 mg, from about 10 mg to about 60 mg, from about 20 mg to about 60 mg, from about 40 mg to about 60 mg, from about 70 mg to about 120 mg, from about 70 mg to about 100 mg, from about 50 mg to about 120 mg, from about 50 mg to 90 mg, from about 30 mg to about 120 mg, from about 30 mg to about 60 mg, from about 30 mg to about 80 mg, from about 30 mg to about 100 mg) of the sodium channel blocker.

In certain embodiments, the sodium channel blocker may be co-administered with Compound 1 or a pharmaceutically acceptable salt thereof to the subject such that the sodium channel blocker (e.g., Formula (I), (I-a), (I-b), (I-c), (II), (III), or (IV), Compound A, or Compound B) is present in an amount ranging from about 0.1 mg/kg to about 1 g/kg, such as from about 0.1 mg/kg to about 10 mg/kg, from about 0.1 mg/kg to about 5 mg/kg, from about 0.1 mg/kg to about 2.5 mg/kg, from about 0.1 mg/kg to about 1.5 mg/kg, from about 0.2 mg/kg to about 15 mg/kg, from about 0.2 mg/kg to about 5 mg/kg, from about 0.5 mg/kg to about 20 mg/kg, from about 0.5 mg/kg to about 10 mg/kg, or from about 0.5 mg/kg to about 5 mg/kg. In some embodiments, the sodium channel blocker, such as Formula I, (I-a), (I-b), (I-c), (II), (III), or (IV), Compound A, or Compound B or a pharmaceutically acceptable salt thereof, or pharmaceutical compositions comprising the same is administered to the subject in an amount ranging from about 10 mg/kg to about 100 mg/kg, such as about 10, 15, 20, 25, 30, 35, 40, 45, 50, 55, 60, 65, 70, 75, 80, 85, 90, 95 or 100 mg/kg. In certain embodiments, the sodium channel blocker, such as Formula I, (I-a), (I-b), (I-c), (II), (III), or (IV), Compound A, or Compound B or a pharmaceutically acceptable salt thereof, or pharmaceutical compositions comprising the same is administered to the subject as a single dose or as multiple doses.

In some embodiments, the dosage form is a liquid form. In some embodiments, the dosage form is in the form of a solution. In some embodiments, the concentration of the sodium channel blocker, such as Formula I, (I-a), (I-b), (I-c), (II), (III), or (IV), Compound A, or Compound B or a pharmaceutically acceptable salt thereof, in the solution is from about 0.1 mg/mL to about 10 mg/mL (e.g., from about 0.5 mg/mL to about 10 mg/mL, from about 1 mg/mL to about 10 mg/mL, from about 2 mg/mL to about 10 mg/mL, from about 3 mg/mL to about 10 mg/mL, from about 4 mg/mL to about 10 mg/mL, from about 5 mg/mL to about 10 mg/mL, from about 6 mg/mL to about 10 mg/mL, from about 0.1 mg/mL to about 8 mg/mL, from about 0.5 mg/mL to about 8 mg/mL, from about 1 mg/mL to about 8 mg/mL, from about 2 mg/mL to about 8 mg/mL, from about 3 mg/mL to about 8 mg/mL, from about 4 mg/mL to about 8 mg/mL, from about 5 mg/mL to about 8 mg/mL, from about 6 mg/mL to about 8 mg/mL, from about 0.5 mg/mL to about 6 mg/mL, from about 1 mg/mL to about 6 mg/mL, from about 2 mg/mL to about 6 mg/mL, from about 3 mg/mL to about 6 mg/mL, from about 4 mg/mL to about 6 mg/mL, from about 0.5 mg/mL to about 4 mg/mL, from about 1 mg/mL to about 4 mg/mL, or from about 2 mg/mL to about 4 mg/mL).

Accordingly, one aspect of the disclosure provides for a composition comprising Compound 1 or a pharmaceutically acceptable salt thereof disclosed herein and at least one additional therapeutic agent, such as a sodium channel blocker (e.g., Formula I, (I-a), (I-b), (I-c), (II), (III), or (IV), Compound A, or Compound B). In some embodiments, the composition comprises Compound 1 or a pharmaceutically acceptable salt thereof disclosed herein and at least two additional therapeutic agents. In some embodiments, the composition comprises Compound 1 or a pharmaceutically acceptable salt thereof disclosed herein and at least three additional therapeutic agents, Compound 1 or a pharmaceutically acceptable salt thereof disclosed herein and at least four additional therapeutic agents, or Compound 1 or a pharmaceutically acceptable salt thereof disclosed herein and at least five additional therapeutic agents.

The methods of combination therapy include co-administration of a single formulation containing Compound 1 or a pharmaceutically acceptable salt thereof disclosed herein and therapeutic agent or agents, essentially contemporaneous administration of a first composition comprising Compound 1 or a pharmaceutically acceptable salt thereof disclosed herein and second composition comprising a therapeutic agent other than Compound 1, such as such as a sodium channel blocker (e.g., Formula I, (I-a), (I-b), (I-c), (II), (III), or (IV), Compound A, or Compound B) and consecutive administration of a first composition comprising Compound 1 or a pharmaceutically acceptable salt thereof disclosed herein and a second composition comprising a therapeutic agent other than Compound 1, in any order, wherein preferably there is a time period where Compound 1 or a pharmaceutically acceptable salt thereof disclosed herein and the therapeutic agent simultaneously exert therapeutic effects.

Other exemplary anti-epilepsy agents that may be therapeutic agents include, for example, brivaracetam, cannabidiol, carbamazepine, clobazam, clonazepam, diazepam, divalproex, eslicarbazepine, ethosuximide, ezogabine, fenfluramine, felbamate, gabapentin, lacosamide, lamotrigine, levetiracetam, lorazepam, neuroactive steroids, oxcarbezepine, permpanel, phenobarbital, phenytoin, pregabalin, primidone, rufinamide, tigabine, topiramate, valproic acid, vigabatrin, and zonisamide.

Anti-migraine agents include, for example, ergots, triptans (sumatriptan, frovatriptan), beta-blockers, tricylic antipressants (amitryptyline), topiramate, and CGRP inhibitors.

### Dosing

In the following embodiments, the dose, dosing frequency, and dosing schedule of a neuroactive steroid is expressed in terms of the dose (e.g., dose of Compound 1) administered. The present disclosure provides methods for treating a neurological disorder, such as epilepsy or an epilepsy syndrome, by administering an effective amount of a neuroactive steroid such as Compound 1 or a pharmaceutically acceptable salt thereof to a patient in need thereof. An effective amount is an amount sufficient to eliminate or significantly reduce the symptoms of the neurological disorder, such as seizure activity, or to alleviate those symptoms.

According to some embodiments of the present disclosure, administering Compound 1 or a pharmaceutically acceptable salt thereof provides a statistically significant therapeutic effect. In one embodiment, the statistically significant therapeutic effect is determined based on one or more standards or criteria provided by one or more regulatory agencies in the United States, e.g., FDA or other countries (such as Australia). In some embodiments, the statistically significant therapeutic effect is determined based on results obtained from regulatory agency approved clinical trial set up and/or procedure.

In some embodiments, the statistically significant therapeutic effect is determined based on a patient population of at least 20, 50, 60, 100, 200, 300, 400, 500, 600, 700, 800, 900, 1000 or 2000. In some embodiments, the statistically significant therapeutic effect is determined based on data obtained from randomized and double-blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%.

In some embodiments, the statistically significant therapeutic effect is determined by a randomized double-blind clinical trial of patients treated with a neuroactive steroid such as Compound 1 or a pharmaceutically acceptable salt thereof and optionally in combination with standard care.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

According to the present disclosure, a neuroactive steroid, such as Compound 1 or a pharmaceutically acceptable salt thereof, is administered on a once or twice a day basis to provide effective relief of the symptoms of the neurological disorder, including for example the symptoms of epilepsy. In some embodiments, a total daily dose of Compound 1 or an equivalent amount of a pharmaceutically acceptable salt thereof is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg.

In certain embodiments, the total daily dose of Compound 1 or a pharmaceutically acceptable salt thereof is from about 5 mg to about 120 mg, including about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges there between. In certain embodiments, the total daily dose of Compound 1 or a pharmaceutically acceptable salt thereof is from about 10 mg to about 60 mg, such as from about 15 mg to about 60 mg. In certain embodiments, the total daily dose of Compound 1 or a pharmaceutically acceptable salt thereof is from about 15 mg to about 80 mg. In certain embodiments, the total daily dose of Compound 1 or a pharmaceutically acceptable salt thereof is from about 15 mg to about 100 mg. In certain embodiments, the total daily dose of Compound 1 or a pharmaceutically acceptable salt thereof is from about 45 mg to about 60 mg. In certain embodiments, the total daily dose of Compound 1 or a pharmaceutically acceptable salt thereof is from about 45 mg to about 80 mg.

In some embodiments, the total daily dose of Compound 1 or a pharmaceutically acceptable salt thereof is at least about 5 mg a day for the treatment of a neurological disorder, such as epilepsy or an epilepsy syndrome. In some embodiments, the total daily dose of Compound 1 or a pharmaceutically acceptable salt thereof is at least about 10 mg a day for the treatment of a neurological disorder, such as epilepsy or an epilepsy syndrome. In some embodiments, the total daily dose of Compound 1 or a pharmaceutically acceptable salt thereof is at least about 15 mg a day for the treatment of a neurological disorder, such as epilepsy or an epilepsy syndrome. In some embodiments, the total daily dose of Compound 1 or a pharmaceutically acceptable salt thereof is at least about 20 mg a day for the treatment of a neurological disorder, such as epilepsy or an epilepsy syndrome. In some embodiments, the total daily dose of Compound 1 or a pharmaceutically acceptable salt thereof is at least about 25 mg a day for the treatment of a neurological disorder, such as epilepsy or an epilepsy syndrome. In some embodiments, the total daily dose of Compound 1 or a pharmaceutically acceptable salt thereof is at least about 30 mg a day for the treatment of a neurological disorder, such as epilepsy or an epilepsy syndrome. In some embodiments, the total daily dose of Compound 1 or a pharmaceutically acceptable salt thereof is at least about 35 mg a day for the treatment of a neurological disorder, such as epilepsy or an epilepsy syndrome. In some embodiments, the total daily dose of Compound 1 or a pharmaceutically acceptable salt thereof is at least about 40 mg a day for the treatment of a neurological disorder, such as epilepsy or an epilepsy syndrome. In some embodiments, the total daily dose of Compound 1 or a pharmaceutically acceptable salt thereof is at least about 45 mg a day for the treatment of a neurological disorder, such as epilepsy or an epilepsy syndrome. In some embodiments, the total daily dose of Compound 1 or a pharmaceutically acceptable salt thereof is at least about 50 mg a day for the treatment of a neurological disorder, such as epilepsy or an epilepsy syndrome. In some embodiments, the total daily dose of Compound 1 or a pharmaceutically acceptable salt thereof is at least about 55 mg a day for the treatment of a neurological disorder, such as epilepsy or an epilepsy syndrome. In some embodiments, the total daily dose of Compound 1 or a pharmaceutically acceptable salt thereof is at least about 60 mg a day for the treatment of a neurological disorder, such as epilepsy or an epilepsy syndrome. In some embodiments, the total daily dose of Compound 1 or a pharmaceutically acceptable salt thereof is at least about 65 mg a day for the treatment of a neurological disorder, such as epilepsy or an epilepsy syndrome. In some embodiments, the total daily dose of Compound 1 or a pharmaceutically acceptable salt thereof is at least about 70 mg a day for the treatment of a neurological disorder, such as epilepsy or an epilepsy syndrome. In some embodiments, the total daily dose of Compound 1 or a pharmaceutically acceptable salt thereof is at least about 75 mg a day for the treatment of a neurological disorder, such as epilepsy or an epilepsy syndrome. In some embodiments, the total daily dose of Compound 1 or a pharmaceutically acceptable salt thereof is at least about 80 mg a day for the treatment of a neurological disorder, such as epilepsy or an epilepsy syndrome. In some embodiments, the total daily dose of Compound 1 or a pharmaceutically acceptable salt thereof is at least about 85 mg a day for the treatment of a neurological disorder, such as epilepsy or an epilepsy syndrome. In some embodiments, the total daily dose of Compound 1 or a pharmaceutically acceptable salt thereof is at least about 90 mg a day for the treatment of a neurological disorder, such as epilepsy or an epilepsy syndrome. In some embodiments, the total daily dose of Compound 1 or a pharmaceutically acceptable salt thereof is at least about 95 mg a day for the treatment of a neurological disorder, such as epilepsy or an epilepsy syndrome. In some embodiments, the total daily dose of Compound 1 or a pharmaceutically acceptable salt thereof is at least about 100 mg a day for the treatment of a neurological disorder, such as epilepsy or an epilepsy syndrome. In some embodiments, the total daily dose of Compound 1 or a pharmaceutically acceptable salt thereof is at least about 105 mg a day for the treatment of a neurological disorder, such as epilepsy or an epilepsy syndrome. In some embodiments, the total daily dose of Compound 1 or a pharmaceutically acceptable salt thereof is at least about 110 mg a day for the treatment of a neurological disorder, such as epilepsy or an epilepsy syndrome. In some embodiments, the total daily dose of Compound 1 or a pharmaceutically acceptable salt thereof is at least about 115 mg a day for the treatment of a neurological disorder, such as epilepsy or an epilepsy syndrome. In some embodiments, the total daily dose of Compound 1 or a pharmaceutically acceptable salt thereof is at least about 120 mg a day for the treatment of a neurological disorder, such as epilepsy or an epilepsy syndrome.

In some embodiments, about 5 mg of Compound 1 or a pharmaceutically acceptable salt thereof once a day is selected to provide a substantial reduction in seizures. In some embodiments, about 5 mg of Compound 1 or a pharmaceutically acceptable salt thereof twice a day is selected to provide a substantial reduction in seizures. In some embodiments, about 10 mg of Compound 1 or a pharmaceutically acceptable salt thereof once a day is selected to provide a substantial reduction in seizures. In some embodiments, about 10 mg of Compound 1 or a pharmaceutically acceptable salt thereof twice a day is selected to provide a substantial reduction in seizures. In some embodiments, about 15 mg of Compound 1 or a pharmaceutically acceptable salt thereof once a day is selected to provide a substantial reduction in seizures. In some embodiments, about 15 mg of Compound 1 or a pharmaceutically acceptable salt thereof twice a day is selected to provide a substantial reduction in seizures. In some embodiments, about 20 mg of Compound 1 or a pharmaceutically acceptable salt thereof once a day is selected to provide a substantial reduction in seizures. In some embodiments, about 20 mg of Compound 1 or a pharmaceutically acceptable salt thereof twice a day is selected to provide a substantial reduction in seizures. In some embodiments, about 25 mg of Compound 1 or a pharmaceutically acceptable salt thereof once a day is selected to provide a substantial reduction in seizures. In some embodiments, about 25 mg of Compound 1 or a pharmaceutically acceptable salt thereof twice a day is selected to provide a substantial reduction in seizures. In some embodiments, about 30 mg of Compound 1 or a pharmaceutically acceptable salt thereof once a day is selected to provide a substantial reduction in seizures. In some embodiments, about 30 mg of Compound 1 or a pharmaceutically acceptable salt thereof twice a day is selected to provide a substantial reduction in seizures. In some embodiments, about 35 mg of Compound 1 or a pharmaceutically acceptable salt thereof once a day is selected to provide a substantial reduction in seizures. In some embodiments, about 35 mg of Compound 1 or a pharmaceutically acceptable salt thereof twice a day is selected to provide a substantial reduction in seizures. In some embodiments, about 40 mg of Compound 1 or a pharmaceutically acceptable salt thereof once a day is selected to provide a substantial reduction in seizures. In some embodiments, about 40 mg of Compound 1 or a pharmaceutically acceptable salt thereof twice a day is selected to provide a substantial reduction in seizures. In some embodiments, about 45 mg of Compound 1 or a pharmaceutically acceptable salt thereof once a day is selected to provide a substantial reduction in seizures. In some embodiments, about 45 mg of Compound 1 or a pharmaceutically acceptable salt thereof twice a day is selected to provide a substantial reduction in seizures. In some embodiments, about 50 mg of Compound 1 or a pharmaceutically acceptable salt thereof once a day is selected to provide a substantial reduction in seizures. In some embodiments, about 50 mg of Compound 1 or a pharmaceutically acceptable salt thereof twice a day is selected to provide a substantial reduction in seizures. In some embodiments, about 55 mg of Compound 1 or a pharmaceutically acceptable salt thereof once a day is selected to provide a substantial reduction in seizures. In some embodiments, about 55 mg of Compound 1 or a pharmaceutically acceptable salt thereof twice a day is selected to provide a substantial reduction in seizures. In some embodiments, about 60 mg of Compound 1 or a pharmaceutically acceptable salt thereof once a day is selected to provide a substantial reduction in seizures. In some embodiments, about 60 mg of Compound 1 or a pharmaceutically acceptable salt thereof twice a day is selected to provide a substantial reduction in seizures. In some embodiments, about 65 mg of Compound 1 or a pharmaceutically acceptable salt thereof once a day is selected to provide a substantial reduction in seizures. In some embodiments, about 70 mg of Compound 1 or a pharmaceutically acceptable salt thereof once a day is selected to provide a substantial reduction in seizures. In some embodiments, about 75 mg of Compound 1 or a pharmaceutically acceptable salt thereof once a day is selected to provide a substantial reduction in seizures. In some embodiments, about 80 mg of Compound 1 or a pharmaceutically acceptable salt thereof once a day is selected to provide a substantial reduction in seizures. In some embodiments, about 85 mg of Compound 1 or a pharmaceutically acceptable salt thereof once a day is selected to provide a substantial reduction in seizures. In some embodiments, about 90 mg of Compound 1 or a pharmaceutically acceptable salt thereof once a day is selected to provide a substantial reduction in seizures. In some embodiments, about 95 mg of Compound 1 or a pharmaceutically acceptable salt thereof once a day is selected to provide a substantial reduction in seizures. In some embodiments, about 100 mg of Compound 1 or a pharmaceutically acceptable salt thereof once a day is selected to provide a substantial reduction in seizures. In some embodiments, about 105 mg of Compound 1 or a pharmaceutically acceptable salt thereof once a day is selected to provide a substantial reduction in seizures. In some embodiments, about 110 mg of Compound 1 or a pharmaceutically acceptable salt thereof once a day is selected to provide a substantial reduction in seizures. In some embodiments, about 115 mg of Compound 1 or a pharmaceutically acceptable salt thereof once a day is selected to provide a substantial reduction in seizures. In some embodiments, about 120 mg of Compound 1 or a pharmaceutically acceptable salt thereof once a day is selected to provide a substantial reduction in seizures. In certain embodiments, the substantial reduction in seizures is a substantial reduction in at least one of tonic-clonic, tonic, clonic, myoclonic, absence, atonic, partial, focal, complex partial, refractory, or febrile seizures.

In some embodiments, the dosing frequency and dose amount per administration of Compound 1 or a pharmaceutically acceptable salt thereof are selected to provide therapeutic effects for the acute treatment of a neurological disorder, such as epilepsy or an epilepsy syndrome. In some embodiments, the dosing frequency and dose amount per administration of Compound 1 or a pharmaceutically acceptable salt thereof are selected to provide therapeutic effects for the acute treatment of a neurological disorder, such as epilepsy or an epilepsy syndrome, and, after the acute treatment of the neurological disorder, Compound 1 or a pharmaceutically acceptable salt thereof is no longer administered and a dosing frequency and dose amount of second anti-epileptic agent is selected to provide therapeutic effects for the chronic treatment of the neurological disorder.

In some embodiments, the dosing frequency and dose amount per administration of Compound 1 or a pharmaceutically acceptable salt thereof are selected to provide therapeutic effects for the acute treatment of a neurological disorder, such as epilepsy or an epilepsy syndrome, and, after the acute treatment of the neurological disorder, a dosing frequency and dose amount of Compound 1 or a pharmaceutically acceptable salt thereof is selected to provide therapeutic effects for the chronic treatment of the neurological disorder. In certain embodiments, the daily dosing of Compound 1 or a pharmaceutically acceptable salt thereof for the acute treatment of the neurological disorder, such as epilepsy or an epilepsy syndrome, is greater than the daily dosing of Compound 1 or a pharmaceutically acceptable salt thereof for the chronic treatment of the neurological disorder.

In some embodiments, the dosing frequency and dose amount per administration of Compound 1 or a pharmaceutically acceptable salt thereof are selected to prevent the recurrence of a neurological disorder, such as epilepsy or an epilepsy syndrome. In some embodiments, the dosing frequency and dose amount per administration of Compound 1 or a pharmaceutically acceptable salt thereof are selected to maintain remission of a neurological disorder, such as epilepsy or an epilepsy syndrome.

In some embodiments, the dosing frequency, dose amount per administration of Compound 1 or a pharmaceutically acceptable salt thereof, and dosing time are selected to provide a therapeutic effect for the treatment of a neurological disorder, such as epilepsy or an epilepsy syndrome and to reduce adverse effects associated with administration of Compound 1 or a pharmaceutically acceptable salt thereof. In some embodiments disclosed herein wherein Compound 1 or a pharmaceutically acceptable salt thereof is administered in the evening, the methods of the disclosure provide reduced somnolence compared to morning administration of Compound 1 or a pharmaceutically acceptable salt thereof. In some embodiments wherein Compound 1 or a pharmaceutically acceptable salt thereof is administered in the evening, the methods of the disclosure provide reduced dizziness compared to morning administration of Compound 1 or a pharmaceutically acceptable salt thereof. In some embodiments wherein Compound 1 or a pharmaceutically acceptable salt thereof is administered in the evening, the methods of the disclosure provide reduced lightheadedness, fatigue, euphoria, feeling drunk, feeling abnormal, headache, cognitive disorder, disturbance of attention, memory impairment, slowed cognition, decreased alertness, constipation, or diarrhea compared to morning administration of Compound 1 or a pharmaceutically acceptable salt thereof.

In certain embodiments, Compound 1 or a pharmaceutically acceptable salt thereof is administered on a once a day or twice a day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In certain embodiments, at least about 5 mg or about 5 mg of Compound 1 or a pharmaceutically acceptable salt thereof is administered on a once a day or twice a day basis for at least a week. In certain embodiments, at least about 10 mg or about 10 mg of Compound 1 or a pharmaceutically acceptable salt thereof is administered on a once a day or twice a day basis for at least a week. In certain embodiments, at least about 15 mg or about 15 mg of Compound 1 or a pharmaceutically acceptable salt thereof is administered on a once a day or twice a day basis for at least a week. In certain embodiments, at least about 20 mg or about 20 mg of Compound 1 or a pharmaceutically acceptable salt thereof is administered on a once a day or twice a day basis for at least a week. In certain embodiments, at least about 25 mg or about 25 mg of Compound 1 or a pharmaceutically acceptable salt thereof is administered on a once a day or twice a day basis for at least a week. In certain embodiments, at least about 30 mg or about 30 mg of Compound 1 or a pharmaceutically acceptable salt thereof is administered on a once a day or twice a day basis for at least a week. In certain embodiments, at least about 35 mg or about 35 mg of Compound 1 or a pharmaceutically acceptable salt thereof is administered on a once a day or twice a day basis for at least a week. In certain embodiments, at least about 40 mg or about 40 mg of Compound 1 or a pharmaceutically acceptable salt thereof is administered on a once a day or twice a day basis for at least a week. In certain embodiments, at least about 45 mg or about 45 mg of Compound 1 or a pharmaceutically acceptable salt thereof is administered on a once a day or twice a day basis for at least a week. In certain embodiments, at least about 50 mg or about 50 mg of Compound 1 or a pharmaceutically acceptable salt thereof is administered on a once a day or twice a day basis for at least a week. In certain embodiments, at least about 55 mg or about 55 mg of Compound 1 or a pharmaceutically acceptable salt thereof is administered on a once a day or twice a day basis for at least a week. In certain embodiments, at least about 60 mg or about 60 mg of Compound 1 or a pharmaceutically acceptable salt thereof is administered on a once a day or twice a day basis for at least a week. In certain embodiments, at least about 65 mg or about 65 mg of Compound 1 or a pharmaceutically acceptable salt thereof is administered on a once a day basis for at least a week. In certain embodiments, at least about 70 mg or about 70 mg of Compound 1 or a pharmaceutically acceptable salt thereof is administered on a once a day basis for at least a week. In certain embodiments, at least about 75 mg or about 75 mg of Compound 1 or a pharmaceutically acceptable salt thereof is administered on a once a day basis for at least a week. In certain embodiments, at least about 80 mg or about 80 mg of Compound 1 or a pharmaceutically acceptable salt thereof is administered on a once a day basis for at least a week. In certain embodiments, at least about 85 mg or about 85 mg of Compound 1 or a pharmaceutically acceptable salt thereof is administered on a once a day basis for at least a week. In certain embodiments, at least about 90 mg or about 90 mg of Compound 1 or a pharmaceutically acceptable salt thereof is administered on a once a day basis for at least a week. In certain embodiments, at least about 95 mg or about 95 mg of Compound 1 or a pharmaceutically acceptable salt thereof is administered on a once a day basis for at least a week. In certain embodiments, at least about 100 mg or about 100 mg of Compound 1 or a pharmaceutically acceptable salt thereof is administered on a once a day basis for at least a week. In certain embodiments, at least about 105 mg or about 105 mg of Compound 1 or a pharmaceutically acceptable salt thereof is administered on a once a day basis for at least a week. In certain embodiments, at least about 110 mg or about 110 mg of Compound 1 or a pharmaceutically acceptable salt thereof is administered on a once a day basis for at least a week. In certain embodiments, at least about 115 mg or about 115 mg of Compound 1 or a pharmaceutically acceptable salt thereof is administered on a once a day basis for at least a week. In certain embodiments, at least about 120 mg or about 120 mg of Compound 1 or a pharmaceutically acceptable salt thereof is administered on a once a day basis for at least a week.

In some embodiments, the method of treating a neurological disorder, such as epilepsy or an epilepsy syndrome, disclosed herein further includes a step of titrating the dose of Compound 1 or a pharmaceutically acceptable salt thereof until a maintenance dose is achieved in the patient. In some embodiments, the titration is conducted for at least about one week until a maintenance dose is achieved in the patient. In one embodiment, the titration is conducted for about 2 weeks until a maintenance dose is achieved in the patient. In some embodiments, the titration is conducted for about 7 days to about 30 days until a maintenance dose is achieved in the patient. In some embodiments, the titration is conducted for about 12 days to about 20 days until a maintenance dose is achieved in the patient. In some embodiments, during the titration step, a constant daily dose of Compound 1 or a pharmaceutically acceptable salt thereof is provided. In further embodiments, the constant daily dose of Compound 1 or a pharmaceutically acceptable salt thereof is provided for at least two weeks.

The daily dose can be titrated in one or more steps. The daily dosage can be titrated by increasing a single daily dosage, or each dose of a twice-daily dosing regimen. The amount a dosage is stepped, where there are multiple titration steps, can be the same, or can be different.

In some embodiments, the titration is initiated with from about 15 mg to about 100 mg of Compound 1 or a pharmaceutically acceptable salt thereof, including about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg including all ranges there between once or twice daily. In some embodiments, the titration is initiated with about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg of Compound 1 or a pharmaceutically acceptable salt thereof once or twice daily. In certain embodiments, doses can be adjusted in 5-30 mg increments every 1 to 4 days. In certain embodiments, doses can be adjusted in 5-30 mg increments every week. In some embodiments, the titration is conducted for at least about one week, 2 weeks, 3 weeks, 4 weeks, 5 weeks, 6 weeks, about 7 weeks, about 8 weeks, about 9 weeks or about 10 weeks prior to the maintenance dose.

In certain embodiments, ascending doses of Compound 1 or a pharmaceutically acceptable salt thereof are administered during the titration until a maintenance dose is achieved in the patient. In certain embodiments, ascending doses of the Compound 1 or a pharmaceutically acceptable salt thereof are administered during the titration until an effective amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg is achieved in the patient.

In certain embodiments, patients are initially administered about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg of Compound 1 or a pharmaceutically acceptable salt once or twice a day and are titrated to a maintenance dose of about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg once or twice a day. In certain embodiments, patients are initially administered from about 15 mg to about 100 mg of Compound 1 or a pharmaceutically acceptable salt, including about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg, including all ranges there between once or twice a day and are titrated to a maintenance dose of from about 20 mg to about 120 mg, including about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg including all ranges therebetween, once or twice a day.

In some embodiments, the present disclosure provides a method of treating a neurological disorder, such as epilepsy or an epilepsy syndrome, that includes the steps of: (a) administering an initial daily dose of Compound 1 or a pharmaceutically acceptable salt thereof for at least one week and (b) administering a maintenance daily dose for at least one week. In certain embodiments, the initial daily dose is greater than the maintenance daily dose. In certain embodiments, the initial daily dose is less than the maintenance daily dose. In certain embodiments, the initial daily dose is administered for two weeks and the maintenance daily dose is administered is administered for at least one month.

In some embodiments, the present disclosure provides a method of treating a neurological disorder, such as epilepsy or an epilepsy syndrome, that includes the steps of:
(a) administering a loading dose of Compound 1 or a pharmaceutically acceptable salt thereof; and
(b) administering a maintenance dose of Compound 1 or a pharmaceutically acceptable salt thereof.

In some embodiments, the loading dose is administered for about 1 day to about 14 days, including about 1 day, about 2 days, about 3 days, about 4 days, about 5 days, about 6 days, about 7 days, about 8 days, about 9 days, about 10 days, about 11 days, about 12 days, about 13 days and about 14 days, including all ranges therebetween. In some embodiments, the loading dose is administered for about 1 day, about 2 days, about 3 days, about 4 days, about 5 days, about 6 days, about 7 days, about 8 days, about 9 days, about 10 days, about 11 days, about 12 days, about 13 days or about 14 days.

In some embodiments, the methods comprise a loading dose of from about 30 mg to about 120 mg, including about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg including all ranges therebetween. In some embodiments, the methods comprise a loading dose of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg,

In some embodiments, the maintenance dose is administered for from about 1 month to about 36 months, including about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months including all ranges there between. In some embodiments, the maintenance dose is administered for about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months.

In some embodiments, the methods comprise a maintenance dose of from about 30 mg to about 120 mg, including about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg including all ranges therebetween, once or twice a day. In some embodiments, the methods comprise a maintenance dose of from about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg, once or twice a day.

In some embodiments, the loading dose administration methods further comprise a cessation period after administration of the loading dose and prior to administration of the maintenance dose.

In some embodiments, the cessation period is about one day, about two days, about three days, about four days, about five days, about six days, or about seven days. In some embodiments, the cessation period is from about one day to about seven days, including about one day, about two days, about three days, about four days, about five days, about six days, and about seven days, including all ranges there between.

In some embodiments, the cessation period is about one week, about two weeks, about three weeks, about four weeks, about five weeks, about six weeks, about seven weeks, or about eight weeks. In some embodiments, the cessation period is from about one week to about eight weeks, including about one week, about two weeks, about three weeks, about four weeks, about five weeks, about six weeks, about seven weeks, and about eight weeks including all ranges there between.

In some embodiments, the methods of the present disclosure comprise continuous administration of Compound 1 or a pharmaceutically acceptable salt thereof for a specified interval (for example, one week) followed by a cessation period wherein the patient is not administered Compound 1 or a pharmaceutically acceptable salt thereof. In some embodiments, the methods of the present disclosure comprise continuous administration of Compound 1 or a pharmaceutically acceptable salt thereof for from about one week to about eight weeks, including about one week, about two weeks, about three weeks, about four weeks, about five weeks, about six weeks, about seven weeks, and about eight weeks including all ranges there between. In some embodiments, the methods of the present disclosure comprise continuous administration of Compound 1 or a pharmaceutically acceptable salt thereof for about one week, about two weeks, about three weeks, about four weeks, about five weeks, about six weeks, about seven weeks, or about eight weeks.

In some embodiments, the methods of the present disclosure comprise continuous administration of Compound 1 or a pharmaceutically acceptable salt thereof for from about one month to about 36 months, including about one month, about two months, about three months, about four months, about five months, about six months, about seven months, about eight months, about 9 months, about 10 months, about 11 months, about 12 months, about 18 months, about 24 months, about 30 months, and about 36 months including all ranges there between. In some embodiments, the methods of the present disclosure comprise continuous administration of Compound 1 or a pharmaceutically acceptable salt thereof for about one month, about two months, about three months, about four months, about five months, about six months, about seven months, about eight months, about nine months, about ten months, about eleven months, about twelve months, about 18 months, about 24 months, about 30 months, or about 36 months.

In some embodiments, the methods of the present disclosure comprise intermittent administration of Compound 1 or a pharmaceutically acceptable salt thereof. As used herein, intermittent administration means cycling a patient in need thereof on and off treatment with Compound 1 or a pharmaceutically acceptable salt thereof for specified time intervals.

In some embodiments, intermittent administration comprises:
(a) administering Compound 1 or a pharmaceutically acceptable salt thereof for a first administration period;
(b) after the first administration period (a), not administering Compound 1 or a pharmaceutically acceptable salt thereof for a cessation period;
(c) after the cessation period (b), administering Compound 1 or a pharmaceutically acceptable salt thereof for a second administration period.

In some embodiments, the intermittent administration further comprises one or more additional cessation periods (for example, a second cessation period) and/or administration periods (for example, a third administration period). In such embodiments, the present disclosure contemplates embodiments wherein the additional cessation and/or administration periods have the durations described herein for the first administration period and the cessation period.

In some embodiments, two or more of the periods (a), (b) and (c) are the same (for example, the first administration period, cessation period and second administration period are each one week). In some embodiments, the periods (a) and (b) (for example, one week) are the same and the period (c) is different (for example, two weeks). In some embodiments, the periods (a) and (c) are the same and the period (b) is different. In some embodiments, the periods (b) and (c) are the same and the period (a) is different. In some embodiments, the periods (a), (b) and (c) are the different (for example, the first administration period is one week, the cessation period is two weeks and the second administration period is three weeks).

In some embodiments, the first administration period is about one week, about two weeks, about three weeks, about four weeks, about five weeks, about six weeks, about seven weeks, or about eight weeks. In some embodiments, the first administration period is from about one week to about eight weeks, including about one week, about two weeks, about three weeks, about four weeks, about five weeks, about six weeks, about seven weeks, and about eight weeks including all ranges there between.

In some embodiments, the cessation period is about one week, about two weeks, about three weeks, about four weeks, about five weeks, about six weeks, about seven weeks, or about eight weeks. In some embodiments, the cessation period is from about one week to about eight weeks, including about one week, about two weeks, about three weeks, about four weeks, about five weeks, about six weeks, about seven weeks, and about eight weeks including all ranges there between.

In some embodiments, the second administration period is about one week, about two weeks, about three weeks, about four weeks, about five weeks, about six weeks, about seven weeks, or about eight weeks. In some embodiments, the second administration period is from about one week to about eight weeks, including about one week, about two weeks, about three weeks, about four weeks, about five weeks, about six weeks, about seven weeks, or about eight weeks including all ranges there between.

In some embodiments, the first administration period, cessation period and second administration period are one week. In some embodiments, the first administration period, cessation period and second administration period are two weeks. In some embodiments, the first administration period, cessation period and second administration period are three weeks. In some embodiments, the first administration period, cessation period and second administration period are four weeks. In some embodiments, the first administration period, cessation period and second administration period are five weeks. In some embodiments, the first administration period, cessation period and second administration period are six weeks. In some embodiments, the first administration period, cessation period and second administration period are seven weeks. In some embodiments, the first administration period, cessation period and second administration period are eight weeks.

In some embodiments, the first administration period is about one week; the cessation period is about three weeks; and the second administration period is about one week.

In some embodiments, the first administration period is about two weeks; the first cessation period is about two weeks; the second administration period is about one week; the second cessation period is about one week and the third administration period is about one week.

In some embodiments, the intermittent administration period (including the administration and cessation periods) is about one month, about two months, about three months, about four months, about five months, about six months, about seven months, about eight months, about nine months, about ten months, about eleven months, about twelve months, about 18 months, about 24 months, about 30 months or about 36 months. In some embodiments, the intermittent administration period is from about one month to about twelve months, including about two months, about three months, about four months, about five months, about six months, about seven months, about eight months, about nine months, about ten months, about eleven months, about twelve months, about 18 months, about 24 months, about 30 months and about 36 months, including all ranges there between.

In some embodiments of the present disclosure, Compound 1 or a pharmaceutically acceptable salt thereof is administered to a patient with food. In some embodiments, Compound 1 or a pharmaceutically acceptable salt thereof is administered to a patient about 5 minutes, about 10 minutes, about 15 minutes, about 30 minutes, about 45 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours or about 8 hours after food is ingested.

In some embodiments, the food ingested is a high fat and high calorie food. In some embodiments, the caloric content of the high fat and high calorie food is at least about 700 kilocalories (kcal), and at least about 40 percent of the caloric content of the food is from fat. For example, the fat can contribute to about 50 percent of the caloric content of the food of high fat and high calorie. In some embodiments, the caloric content of the high fat and high calorie food is about 900 kilocalories.

In some embodiments, the food ingested is a medium fat and medium calorie food. In some embodiments, the caloric content of the medium fat and medium calorie food is about 300 kcal to about 700 kcal, and between about 20 percent to about 40 percent of the caloric content of the food is from fat. In some embodiments, the caloric content of the medium fat and medium calorie food is about 400 kcal.

In some embodiments, the food ingested is a low fat and low calorie food. In some embodiments, the caloric content of the low fat and low calorie food is between about 100 kcal to about 300 kcal, and the fat content is approximately 3 grams or less, or about 20 percent or less of the caloric content of the food are from fat. In some embodiments, the caloric content of the food of low fat and low calorie is about 100 kilocalories.

In some embodiments of the present disclosure, Compound 1 or a pharmaceutically acceptable salt thereof is administered to a patient after a fasting period. In some embodiments, Compound 1 or a pharmaceutically acceptable salt thereof is administered to a patient after a fasting period of about 1 hour, about 2 hours, about 3 hours, about 4 hours, about 5 hours, about 6 hours, about 7 hours, about 8 hours, about 9 hours, about 10 hours, about 11 hours, or about 12 hours. In some embodiments, Compound 1 or a pharmaceutically acceptable salt thereof is administered on an empty stomach.

In some embodiments of the present disclosure, Compound 1 or a pharmaceutically acceptable salt thereof is administered to a patient without regard to meals. In some embodiments, Compound 1 or a pharmaceutically acceptable salt thereof is administered to a patient with or without a meal.

In some embodiments, Compound 1 or a pharmaceutically acceptable salt thereof is administered to a patient in the evening. In some embodiments, administration in the evening occurs at bedtime. In some embodiments, Compound 1 or a pharmaceutically acceptable salt thereof is administered to a patient at bedtime. In some embodiments, Compound 1 or a pharmaceutically acceptable salt thereof is administered to a patient prior to bedtime. A patient's bedtime may be at any time of the day, for example, at about 12:00 a.m., at about 12:30 a.m., at about 1:00 a.m., at about 1:30 a.m., at about 2:00 a.m., at about 2:30 a.m., at about 3:00 a.m., at about 3:30 a.m., at about 4:00 a.m., at about 4:30 a.m., at about 5:00 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., at about 12:00 p.m., at about 12:30 p.m., at about 1:00 p.m., at about 1:30 p.m., at about 2:00 p.m., at about 2:30 p.m., at about 3:00 p.m., at about 3:30 p.m., at about 4:00 p.m., at about 4:30 p.m., at about 5:00 p.m., at about 5:30 p.m., at about 6:00 p.m., at about 6:30 p.m., at about 7:00 p.m., at about 7:30 p.m., at about 8:00 p.m., at about 8:30 p.m., at about 9:00 p.m., at about 9:30 p.m., at about 10:00 p.m., at about 10:30 p.m., at about 11:00 p.m., or at about 11:30 p.m.

In some embodiments, the administration in the evening occurs prior to bedtime. In some embodiments, administration in the evening occurs within about three hours of bedtime, for example, about three hours prior to bedtime, about 2.5 hours prior to bedtime, about 2 hours prior to bedtime, about 1.5 hours prior to bedtime, about 1 hour prior to bedtime, about 30 minutes prior to bedtime, about 20 minutes prior to bedtime, about 15 minutes prior to bedtime, about 10 minutes prior to bedtime, or about 5 minutes prior to bedtime. In some embodiments, administration in the evening occurs within about two hours prior to bedtime.

In some embodiments, administration in the evening occurs between about 5 p.m. and about 12 a.m., for example, at about 5 p.m., at about 5:30 p.m., at about 6:00 p.m., at about 6:30 p.m., at about 7:00 p.m., at about 7:30 p.m., at about 8:00 p.m., at about 8:30 p.m., at about 9:00 p.m., at about 9:30 p.m., at about 10:00 p.m., at about 10:30 p.m., at about 11:00 p.m., at about 11:30 p.m., or at about 12:00 a.m.

In some embodiments, administration in the evening occurs after 8 p.m., for example, at about 8 p.m., at about 8:30 p.m., at about 9:00 p.m., at about 9:30 p.m., at about 10:00 p.m., at about 10:30 p.m., at about 11:00 p.m., at about 11:30 p.m., at about 12:00 a.m., at about 12:30 a.m., at about 1:00 a.m., at about 1:30 a.m., or about 2:00 a.m.

In some embodiments, administration in the evening occurs between about 5 p.m. and bedtime.

In some embodiments, administration in the evening occurs with the evening meal. In some embodiments, administration in the evening occurs within about six hours of the evening meal. In some embodiments, administration in the evening occurs within about five hours of the evening meal. In some embodiments, administration in the evening occurs within about four hours of the evening meal. In some embodiments, administration in the evening occurs within about three hours of the evening meal. In some embodiments, administration in the evening occurs within about two hours of the evening meal. In some embodiments, administration in the evening occurs within about one hour of the evening meal. In some embodiments, administration in the evening occurs within about 30 minutes of the evening meal. In some embodiments, administration in the evening occurs two hours after the evening meal. In some embodiments, administration in the evening occurs four hours after the evening meal. In some embodiments, administration in the evening occurs after the evening meal. In some embodiments, the evening meal is dinner. In some embodiments, administration in the evening occurs two hours after dinner. In some embodiments, administration in the evening occurs four hours after dinner. In some embodiments, administration in the evening occurs after dinner.

In some embodiments, Compound 1 or a pharmaceutically acceptable salt thereof is administered to a patient in the morning. In some embodiments, a morning dose is administered when a patient wakes up. A patient may wake up at any time of the day, for example, at about 12:00 a.m., at about 12:30 a.m., at about 1:00 a.m., at about 1:30 a.m., at about 2:00 a.m., at about 2:30 a.m., at about 3:00 a.m., at about 3:30 a.m., at about 4:00 a.m., at about 4:30 a.m., at about 5:00 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., at about 12:00 p.m., at about 12:30 p.m., at about 1:00 p.m., at about 1:30 p.m., at about 2:00 p.m., at about 2:30 p.m., at about 3:00 p.m., at about 3:30 p.m., at about 4:00 p.m., at about 4:30 p.m., at about 5:00 p.m., at about 5:30 p.m., at about 6:00 p.m., at about 6:30 p.m., at about 7:00 p.m., at about 7:30 p.m., at about 8:00 p.m., at about 8:30 p.m., at about 9:00 p.m., at about 9:30 p.m., at about 10:00 p.m., at about 10:30 p.m., at about 11:00 p.m., at about 11:30 p.m. In some embodiments, administration in the morning occurs within about three hours after a patient wakes up, for example, about three hours after a patient wakes up, about 2.5 hours after a patient wakes up, about 2 hours after a patient wakes up, about 1.5 hours after a patient wakes up, about 1 hour after a patient wakes up, about 30 minutes after a patient wakes up, about 20 minutes after a patient wakes up, about 15 minutes after a patient wakes up, about 10 minutes after a patient wakes up, or about 5 minutes after a patient wakes up. In some embodiments, administration in the morning occurs within about two hours after a patient wakes up. In some embodiments, Compound 1 or a pharmaceutically acceptable salt thereof is administered in the morning after a period of fasting.

In some embodiments, administration in the morning occurs between about 5 a.m. and about 12 p.m., for example, at about 5 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., or at about 12:00 p.m.

In some embodiments, administration in the morning occurs at or after 5:00 a.m., for example, at about 5:00 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., or at about 12:00 p.m.

In some embodiments, administration in the morning occurs between about 5 a.m. and 12:00 p.m., for example, at about 5:00 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., or at about 12:00 p.m.

In some embodiments, administration in the morning occurs with the morning meal. In some embodiments, administration in the morning occurs within about six hours of the morning meal. In some embodiments, administration in the morning occurs within about five hours of the morning meal. In some embodiments, administration in the morning occurs within about four hours of the morning meal. In some embodiments, administration in the morning occurs within about three hours of the morning meal. In some embodiments, administration in the morning occurs within about two hours of the morning meal. In some embodiments, administration in the morning occurs within about one hour of the morning meal. In some embodiments, administration in the morning occurs within about 30 minutes of the morning meal.

In some embodiments, administration in the morning occurs between about eight hours and about 16 hours before administration of an evening dose. In some embodiments, administration in the morning occurs between about ten hours and about 14 hours before administration of an evening dose. In some embodiments, administration in the morning occurs about eight hours before administration of an evening dose. In some embodiments, administration in the morning occurs about nine hours before administration of an evening dose. In some embodiments, administration in the morning occurs about ten hours before administration of an evening dose. In some embodiments, administration in the morning occurs about 11 hours before administration of an evening dose. In some embodiments, administration in the morning occurs about 12 hours before administration of an evening dose. In some embodiments, administration in the morning occurs about 13 hours before administration of an evening dose. In some embodiments, administration in the morning occurs about 14 hours before administration of an evening dose. In some embodiments, administration in the morning occurs about 15 hours before administration of an evening dose. In some embodiments, administration in the morning occurs about 16 hours before administration of an evening dose.

In some embodiments, the methods of the present disclosure comprise controlling the gastrointestinal pH of the patient prior to, concurrently with or after administration of Compound 1 or a pharmaceutically acceptable salt thereof. In some embodiments, the gastrointestinal pH of the patient is controlled prior to administration of Compound 1 or a pharmaceutically acceptable salt thereof. In some embodiments, the gastrointestinal pH of the patient is controlled after administration of Compound 1 or a pharmaceutically acceptable salt thereof.

In some embodiments, the pH is controlled by administering a drug, food or liquid to a patient that decreases gastrointestinal pH prior to, concurrently with or after administration of Compound 1 or a pharmaceutically acceptable salt thereof. In some embodiments, the liquid is an acidic beverage (such as a carbonated beverage).

In some embodiments, the pH is controlled by the patient avoiding a drug, food or beverage that increases gastrointestinal pH prior to, concurrently with or after administration of Compound 1 or a pharmaceutically acceptable salt thereof. In some embodiments, the drug that increases gastrointestinal pH is a proton pump inhibitor or an orally-administered antacid.

In certain embodiments, the initial daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg and the maintenance daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, or about 115 mg provided the initial daily dose is greater than the maintenance daily dose.

According to some embodiments, the substantial reduction in seizures provided by the methods of the present disclosure requires treatment for a specified time interval (e.g., at least one week) before the patient experiences substantial reduction of seizures (i.e., there is an induction period before the patient experiences a substantial reduction in seizures). In some embodiments, after treatment for at least one week, at least two weeks, at least three weeks, at least four weeks, at least five weeks, at least six weeks, at least seven weeks or at least eight weeks, the patient experiences a substantial reduction of seizures compared to prior to the treatment. In certain embodiments, after treatment for at least one week the patient experiences a substantial reduction of seizures compared to prior to the treatment.

According to some embodiments of the present disclosure, the dosing frequency, dosing schedule and dose amount per administration of Compound 1 or a pharmaceutically acceptable salt thereof are selected to reduce or ameliorate adverse effects associated with the administration of Compound 1 or pharmaceutically acceptable salt thereof for the treatment of a neurological disorder, such as epilepsy or an epilepsy syndrome.

In some embodiments, the adverse event is somnolence or dizziness. In some embodiments, the adverse event is sedation, lightheadedness, fatigue, euphoria, feeling drunk, feeling abnormal, headache, cognitive disorder, disturbance of attention, memory impairment, slowed cognition, decreased alertness, constipation, or diarrhea.

According to some embodiments of the present disclosure, the dosing frequency and dose amount per administration of Compound 1 or a pharmaceutically acceptable salt thereof are selected to provide therapeutic effects for the treatment of a neurological disorder, such as epilepsy or an epilepsy syndrome.

According to some embodiments of the present disclosure, the dosing frequency and dose amount per administration of Compound 1 are selected to provide therapeutic effects for the treatment of a neurological disorder, such as epilepsy or an epilepsy syndrome that is refractory to other treatments (e.g., treatment-resistant epilepsy).

According to some embodiments of the present disclosure, the dosing frequency and dose amount per administration of Compound 1 or a pharmaceutically acceptable salt thereof are selected to provide therapeutic effects for the treatment of a neurological disorder, such as epilepsy or an epilepsy syndrome that is partially responsive to other therapies. According to some embodiments of the present disclosure, the dosing frequency and dose amount per administration of Compound 1 or a pharmaceutically acceptable salt thereof are selected to provide an adjunctive treatment for a neurological disorder, such as epilepsy or an epilepsy syndrome.

In some embodiments, after the treatment the patient experiences a substantial reduction of seizures that is characterized by at least about a 10% decline in the total number of seizures experienced compared to prior to the treatment. In some embodiments, the reduction of seizures is characterized by a decline in the total number of seizures experienced compared to prior to the treatment ranging from about 10% to about 100%, for example, about 20%, about 30%, about 40%, about 50%, about 60%, about 70%, about 80%, about 90%, and about 100%, compared to prior to the treatment.

According to some embodiments of the present disclosure, the methods of the present disclosure provide therapeutically effective blood plasma levels of Compound 1 for treating a neurological disorder, such as epilepsy or an epilepsy syndrome. Blood plasma levels of Compound 1 may be expressed using pharmacokinetic parameters that are known to those skilled in the art, such as steady state plasma levels, AUC, Cmax, tₘₐₓ, and Cmin. Throughout the present disclosure pharmacokinetic parameters are described in terms of providing a steady state plasma level of a particular PK parameter (such as steady state plasma Cₘₐₓ, steady state AUC, etc.). However, the present disclosure contemplates embodiments where the steady state PK parameters that are expressed herein are average values from a patient population (such as a mean value). Thus, the following description of pharmacokinetic parameters describes mean steady state PK parameter values as well as values from an individual patient.

In some embodiments, the present methods provide steady state plasma levels of Compound 1 that correlate to one or more statistically significant therapeutic effects. In certain embodiments, the therapeutically effective steady state plasma levels of Compound 1 provided by the methods of the present disclosure range from about 1 ng/ml to about 500 ng/ml, including about 1 ng/ml, about 5 ng/mL, about 10 ng/mL, about 15 ng/ml, about 20 ng/ml, about 25 ng/mL, about 30 ng/mL, about 35 ng/mL, about 40 ng/ml, about 45 ng/ml, about 50 ng/ml, about 55 ng/mL, about 60 ng/ml, about 65 ng/ml, about 70 ng/ml, about 75 ng/mL about 80 ng/mL, about 85 ng/mL, about 90 ng/ml, about 95 ng/mL, about 100 ng/mL, about 105 ng/mL, about 110 ng/mL, about 115 ng/mL, about 120 ng/ml, about 125 ng/mL, about 130 ng/mL, about 135 ng/mL, about 140 ng/mL, about 145 ng/mL, about 150 ng/mL, about 155 ng/mL, about 160 ng/mL, about 165 ng/ml, about 170 ng/mL, about 175 ng/mL about 180 ng/mL, about 185 ng/ml, about 190 ng/ml, about 195 ng/mL, about 200 ng/mL, about 205 ng/mL, about 210 ng/mL, about 215 ng/mL, about 220 ng/ml, about 225 ng/ml, about 230 ng/mL, about 235 ng/mL, about 240 ng/mL, about 245 ng/mL, about 250 ng/mL, about 255 ng/mL, about 260 ng/mL, about 265 ng/mL, about 270 ng/mL, about 275 ng/ml, about 280 ng/mL, about 285 ng/mL, about 290 ng/mL, about 295 ng/mL, about 300 ng/ml, about 305 ng/mL, about 310 ng/mL, about 315 ng/mL, about 320 ng/mL, about 325 ng/mL, about 330 ng/mL, about 335 ng/mL, about 340 ng/mL, about 345 ng/mL, about 350 ng/ml, about 355 ng/mL, about 360 ng/mL, about 365 ng/mL, about 370 ng/ml, about 375 ng/ml, about 380 ng/mL, about 385 ng/mL, about 390 ng/mL, about 395 ng/ml, about 400 ng/ml, about 405 ng/mL, about 410 ng/mL, about 415 ng/mL, about 420 ng/mL, about 425 ng/mL, about 430 ng/mL, about 435 ng/mL, about 440 ng/mL, about 445 ng/mL, about 450 ng/ml, about 455 ng/mL, about 460 ng/mL, about 465 ng/mL, about 470 ng/ml, about 475 ng/ml, about 480 ng/ml, about 485 ng/mL, about 490 ng/mL, about 495 ng/mL, and about 500 ng/mL, including all ranges there between. In certain embodiments, the therapeutically effective steady state plasma levels of Compound 1 provided by the methods of the present disclosure range from about 50 ng/ml to 200 ng/ml.

In certain embodiments, the therapeutically effective steady state plasma levels of Compound 1 is provided by administering a daily dose of Compound 1 or a pharmaceutically acceptable salt thereof of about 15 mg. In further embodiments, the therapeutically effective steady state plasma levels of Compound 1 is provided by administering about 15 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 20 mg. In further embodiments, the therapeutically effective steady state plasma levels of Compound 1 are provided by administering about 10 mg of Compound 1 or a pharmaceutically acceptable salt thereof twice a day. In further embodiments, the therapeutically effective steady state plasma levels of Compound 1 are provided by administering about 20 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 25 mg. In further embodiments, the therapeutically effective steady state plasma levels of Compound 1 is provided by administering about 25 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 30 mg. In further embodiments, the therapeutically effective steady state plasma levels of Compound 1 are provided by administering about 15 mg of Compound 1 or a pharmaceutically acceptable salt thereof twice a day. In further embodiments, the therapeutically effective steady state plasma levels of Compound 1 are provided by administering about 30 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 35 mg. In further embodiments, the therapeutically effective steady state plasma levels of Compound 1 are provided by administering about 35 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 40 mg. In further embodiments, the therapeutically effective steady state plasma levels of Compound 1 are provided by administering about 20 mg of Compound 1 or a pharmaceutically acceptable salt thereof twice a day. In further embodiments, the therapeutically effective steady state plasma levels of Compound 1 are provided by administering about 40 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 45 mg. In further embodiments, the therapeutically effective steady state plasma levels of Compound 1 are provided by administering about 45 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 50 mg. In further embodiments, the therapeutically effective steady state plasma levels of Compound 1 are provided by administering about 25 mg of Compound 1 or a pharmaceutically acceptable salt thereof twice a day. In further embodiments, the therapeutically effective steady state plasma levels of Compound 1 are provided by administering about 50 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 55 mg. In further embodiments, the therapeutically effective steady state plasma levels of Compound 1 are provided by administering about 55 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 60 mg. In further embodiments, the therapeutically effective steady state plasma levels of Compound 1 are provided by administering about 30 mg of Compound 1 or a pharmaceutically acceptable salt thereof twice a day. In further embodiments, the therapeutically effective steady state plasma levels of Compound 1 are provided by administering about 60 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 65 mg. In further embodiments, the therapeutically effective steady state plasma levels of Compound 1 are provided by administering about 65 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 70 mg. In further embodiments, the therapeutically effective steady state plasma levels of Compound 1 are provided by administering about 35 mg of Compound 1 or a pharmaceutically acceptable salt thereof twice a day. In further embodiments, the therapeutically effective steady state plasma levels of Compound 1 are provided by administering about 70 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 75 mg. In further embodiments, the therapeutically effective steady state plasma levels of Compound 1 are provided by administering about 75 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 80 mg. In further embodiments, the therapeutically effective steady state plasma levels of Compound 1 are provided by administering about 40 mg of Compound 1 or a pharmaceutically acceptable salt thereof twice a day. In further embodiments, the therapeutically effective steady state plasma levels of Compound 1 are provided by administering about 80 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 85 mg. In further embodiments, the therapeutically effective steady state plasma levels of Compound 1 are provided by administering about 85 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 90 mg. In further embodiments, the therapeutically effective steady state plasma levels of Compound 1 are provided by administering about 45 mg of Compound 1 or a pharmaceutically acceptable salt thereof twice a day. In further embodiments, the therapeutically effective steady state plasma levels of Compound 1 are provided by administering about 90 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 95 mg. In further embodiments, the therapeutically effective steady state plasma levels of Compound 1 are provided by administering about 95 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 100 mg. In further embodiments, the therapeutically effective steady state plasma levels of Compound 1 are provided by administering about 50 mg of Compound 1 or a pharmaceutically acceptable salt thereof twice a day. In further embodiments, the therapeutically effective steady state plasma levels of Compound 1 are provided by administering about 100 mg of Compound 1 or a pharmaceutically acceptable salt thereof once a day.

In some embodiments, the present methods provide mean steady state AUC₀₋₂₄ₕ (expressed in terms of ng*hr/mL) levels of Compound 1 that correlate to one or more statistically significant therapeutic effects. In certain embodiments, the therapeutically effective mean steady state AUC₀₋₂₄ₕ levels of Compound 1 provided by the methods of the present disclosure range from about 50 ng*hr/mL to about 2300 ng*hr/mL, including about 50 ng*hr/mL, 100 ng*hr/mL, 150 ng*hr/mL, 200 ng*hr/mL, 250 ng*hr/mL, 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL and about 2300 ng*hr/mL, including all ranges there between. In certain embodiments, the therapeutically effective mean steady state AUC₀₋₂₄ₕ levels of Compound 1 provided by the methods of the present disclosure range from about 500 ng*hr/mL to about 1000 ng*hr/mL, including about 550 ng*hr/mL, about 600 ng*hr/mL, about 650 ng*hr/mL, about 700 ng*hr/mL, about 750 ng*hr/mL, about 800 ng*hr/mL, about 850 ng*hr/mL and about 900 ng*hr/mL, including all ranges there between. In certain embodiments, the therapeutically effective mean steady state AUC₀₋₂₄ₕ levels of Compound 1 provided by the methods of the present disclosure range from about 600 ng*hr/mL to about 900 ng*hr/mL.

In certain embodiments, the mean steady state AUC₀₋₂₄ₕ of Compound 1 is provided by administering a daily dose of Compound 1 or a pharmaceutically acceptable salt thereof of about 15 mg. In further embodiments, the mean steady state AUC₀₋₂₄ₕ of Compound 1 is provided by administering about 15 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 20 mg. In further embodiments, the mean steady state AUC₀₋₂₄ₕ of Compound 1 is provided by administering about 10 mg of Compound 1 or a pharmaceutically acceptable salt thereof twice a day. In further embodiments, the mean steady state AUC₀₋₂₄ₕ of Compound 1 is provided by administering about 20 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 25 mg. In further embodiments, the mean steady state AUC₀₋₂₄ₕ of Compound 1 is provided by administering about 25 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 30 mg. In further embodiments, the mean steady state AUC₀₋₂₄ₕ of Compound 1 is provided by administering about 15 mg of Compound 1 or a pharmaceutically acceptable salt thereof twice a day. In further embodiments, the mean steady state AUC₀₋₂₄ₕ of Compound 1 is provided by administering about 30 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 35 mg. In further embodiments, the mean steady state AUC₀₋₂₄ₕ of Compound 1 is provided by administering about 35 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 40 mg. In further embodiments, the mean steady state AUC₀₋₂₄ₕ of Compound 1 is provided by administering about 20 mg of Compound 1 or a pharmaceutically acceptable salt thereof twice a day. In further embodiments, the mean steady state AUC₀₋₂₄ₕ of Compound 1 is provided by administering about 40 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 45 mg. In further embodiments, the mean steady state AUC₀₋₂₄ₕ of Compound 1 is provided by administering about 45 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 50 mg. In further embodiments, the mean steady state AUC₀₋₂₄ₕ of Compound 1 is provided by administering about 25 mg of Compound 1 or a pharmaceutically acceptable salt thereof twice a day. In further embodiments, the mean steady state AUC₀₋₂₄ₕ of Compound 1 is provided by administering about 50 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 55 mg. In further embodiments, the mean steady state AUC₀₋₂₄ₕ of Compound 1 is provided by administering about 55 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 60 mg. In further embodiments, the mean steady state AUC₀₋₂₄ₕ of Compound 1 is provided by administering about 30 mg of Compound 1 or a pharmaceutically acceptable salt thereof twice a day. In further embodiments, the mean steady state AUC₀₋₂₄ₕ of Compound 1 is provided by administering about 60 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 65 mg. In further embodiments, the mean steady state AUC₀₋₂₄ₕ of Compound 1 is provided by administering about 65 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 70 mg. In further embodiments, the mean steady state AUC₀₋₂₄ₕ of Compound 1 is provided by administering about 35 mg of Compound 1 or a pharmaceutically acceptable salt thereof twice a day. In further embodiments, the mean steady state AUC₀₋₂₄ₕ of Compound 1 is provided by administering about 70 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 75 mg. In further embodiments, the mean steady state AUC₀₋₂₄ₕ of Compound 1 is provided by administering about 75 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 80 mg. In further embodiments, the mean steady state AUC₀₋₂₄ₕ of Compound 1 is provided by administering about 40 mg of Compound 1 or a pharmaceutically acceptable salt thereof twice a day. In further embodiments, the mean steady state AUC₀₋₂₄ₕ of Compound 1 is provided by administering about 80 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 85 mg. In further embodiments, the mean steady state AUC₀₋₂₄ₕ of Compound 1 is provided by administering about 85 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 90 mg. In further embodiments, the mean steady state AUC₀₋₂₄ₕ of Compound 1 is provided by administering about 45 mg of Compound 1 or a pharmaceutically acceptable salt thereof twice a day. In further embodiments, the mean steady state AUC₀₋₂₄ₕ of Compound 1 is provided by administering about 90 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 95 mg. In further embodiments, the mean steady state AUC₀₋₂₄ₕ of Compound 1 is provided by administering about 95 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 100 mg. In further embodiments, the mean steady state AUC₀₋₂₄ₕ of Compound 1 is provided by administering about 50 mg of Compound 1 or a pharmaceutically acceptable salt thereof twice a day. In further embodiments, the mean steady state AUC₀₋₂₄ₕ of Compound 1 is provided by administering about 100 mg of Compound 1 or a pharmaceutically acceptable salt thereof once a day. In some embodiments, the present methods provide an area under the concentration time curve from time zero to infinity AUC_{inf} (expressed in terms of ng*hr/mL) levels of Compound 1 that correlate to one or more statistically significant therapeutic effects. In certain embodiments, the therapeutically effective mean steady state AUC_{inf} levels of Compound 1 provided by the methods of the present disclosure range from about 1000 ng*hr/mL to about 4000 ng*hr/mL, including about 1000 ng*hr/mL, about 1050 ng*hr/mL, about 1100 ng*hr/mL, about 1150 ng*hr/mL, about 1200 ng*hr/mL, about 1250 ng*hr/mL, about 1300 ng*hr/mL, about 1350 ng*hr/mL, about 1400 ng*hr/mL, about 1450 ng*hr/mL, about 1500 ng*hr/mL, about 1550 ng*hr/mL, about 1600 ng*hr/mL, about 1650 ng*hr/mL, about 1700 ng*hr/mL, about 1750 ng*hr/mL, about 1800 ng*hr/mL, about 1850 ng*hr/mL, about 1900 ng*hr/mL, about 1950 ng*hr/mL, about 2000 ng*hr/mL, about 2050 ng*hr/mL, about 2100 ng*hr/mL, about 2150 ng*hr/mL, about 2200 ng*hr/mL, about 2250 ng*hr/mL, about 2300 ng*hr/mL, about 2350 ng*hr/mL, about 2400 ng*hr/mL, about 2450 ng*hr/mL, about 2500 ng*hr/mL, about 2550 ng*hr/mL, about 2600 ng*hr/mL, about 2650 ng*hr/mL, about 2700 ng*hr/mL, about 2750 ng*hr/mL, about 2800 ng*hr/mL, about 2850 ng*hr/mL, about 2900 ng*hr/mL, about 2950 ng*hr/mL, about 3000 ng*hr/mL, about 3050 ng*hr/mL, about 3100 ng*hr/mL, about 3150 ng*hr/mL, about 3200 ng*hr/mL, about 3250 ng*hr/mL, about 3300 ng*hr/mL, about 3350 ng*hr/mL, about 3400 ng*hr/mL, about 3450 ng*hr/mL, about 3500 ng*hr/mL, about 3550 ng*hr/mL, about 3600 ng*hr/mL, about 3650 ng*hr/mL, about 3700 ng*hr/mL, about 3750 ng*hr/mL, about 3800 ng*hr/mL, about 3850 ng*hr/mL, about 3900 ng*hr/mL, about 3950 ng*hr/mL, about 4000 ng*hr/mL, including all ranges there between.

In certain embodiments, the therapeutically effective AUC_{inf} of Compound 1 provided by the methods of the present disclosure ranges from about 2500 ng*hr/mL to about 3500 ng*hr/mL, including about 2500 ng*hr/mL, about 2550 ng*hr/mL, about 2600 ng*hr/mL, about 2650 ng*hr/mL, about 2700 ng*hr/mL, about 2750 ng*hr/mL, about 2800 ng*hr/mL, about 2850 ng*hr/mL, about 2900 ng*hr/mL, about 2950 ng*hr/mL, about 3000 ng*hr/mL, about 3050 ng*hr/mL, about 3100 ng*hr/mL, about 3150 ng*hr/mL, about 3200 ng*hr/mL, about 3250 ng*hr/mL, about 3300 ng*hr/mL, about 3350 ng*hr/mL, about 3400 ng*hr/mL, about 3450 ng*hr/mL, about 3500 ng*hr/mL, including all ranges there between. In certain embodiments, the therapeutically effective mean steady state AUC_{inf} of Compound 1 provided by the methods of the present disclosure ranges from about 1500 ng*hr/mL to about 2500 ng*hr/mL, including about 1500 ng*hr/mL, about 1550 ng*hr/mL, about 1600 ng*hr/mL, about 1650 ng*hr/mL, about 1700 ng*hr/mL, about 1750 ng*hr/mL, about 1800 ng*hr/mL, about 1850 ng*hr/mL, about 1900 ng*hr/mL, about 1950 ng*hr/mL, about 2000 ng*hr/mL, about 2050 ng*hr/mL, about 2100 ng*hr/mL, about 2150 ng*hr/mL, about 2200 ng*hr/mL, about 2250 ng*hr/mL, about 2300 ng*hr/mL, about 2350 ng*hr/mL, about 2400 ng*hr/mL, about 2450 ng*hr/mL, about 2500 ng*hr/mL, including all ranges there between.

In certain embodiments, the AUC_{inf} of Compound 1 is provided by administering a daily dose of Compound 1 or a pharmaceutically acceptable salt thereof of about 15 mg. In further embodiments, the AUC_{inf} levels of Compound 1 is provided by administering about 15 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 20 mg. In further embodiments, the mean steady state AUC_{inf} of Compound 1 is provided by administering about 10 mg of Compound 1 or a pharmaceutically acceptable salt thereof twice a day. In further embodiments, the mean steady state AUC_{inf} of Compound 1 is provided by administering about 20 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 25 mg. In further embodiments, the AUC_{inf} of Compound 1 is provided by administering about 25 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 30 mg. In further embodiments, the AUC_{inf} of Compound 1 is provided by administering about 15 mg of Compound 1 or a pharmaceutically acceptable salt thereof twice a day. In further embodiments, the AUC_{inf} of Compound 1 is provided by administering about 30 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 35 mg. In further embodiments, the AUC_{inf} of Compound 1 is provided by administering about 35 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 40 mg. In further embodiments, the AUC_{inf} of Compound 1 is provided by administering about 20 mg of Compound 1 or a pharmaceutically acceptable salt thereof twice a day. In further embodiments, the AUC_{inf} of Compound 1 is provided by administering about 40 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 45 mg. In further embodiments, the AUC_{inf} of Compound 1 is provided by administering about 45 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 50 mg. In further embodiments, the AUC_{inf} of Compound 1 is provided by administering about 25 mg of Compound 1 or a pharmaceutically acceptable salt thereof twice a day. In further embodiments, the AUC_{inf} of Compound 1 is provided by administering about 50 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 55 mg. In further embodiments, the AUC_{inf} of Compound 1 is provided by administering about 55 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 60 mg. In further embodiments, the AUC_{inf} of Compound 1 is provided by administering about 30 mg of Compound 1 or a pharmaceutically acceptable salt thereof twice a day. In further embodiments, the AUC_{inf} of Compound 1 is provided by administering about 60 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 65 mg. In further embodiments, the AUC_{inf} of Compound 1 is provided by administering about 65 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 70 mg. In further embodiments, the AUC_{inf} of Compound 1 is provided by administering about 35 mg of Compound 1 or a pharmaceutically acceptable salt thereof twice a day. In further embodiments, the AUC_{inf} of Compound 1 is provided by administering about 70 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 75 mg. In further embodiments, the AUC_{inf} of Compound 1 is provided by administering about 75 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 80 mg. In further embodiments, the AUC_{inf} of Compound 1 is provided by administering about 40 mg of Compound 1 or a pharmaceutically acceptable salt thereof twice a day. In further embodiments, the AUC_{inf} of Compound 1 is provided by administering about 80 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 85 mg. In further embodiments, the AUC_{inf} of Compound 1 is provided by administering about 85 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 90 mg. In further embodiments, the AUC_{inf} of Compound 1 is provided by administering about 45 mg of Compound 1 or a pharmaceutically acceptable salt thereof twice a day. In further embodiments, the AUC_{inf} of Compound 1 is provided by administering about 90 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 95 mg. In further embodiments, the AUC_{inf} of Compound 1 is provided by administering about 95 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 100 mg. In further embodiments, the AUC_{inf} of Compound 1 is provided by administering about 50 mg of Compound 1 or a pharmaceutically acceptable salt thereof twice a day. In further embodiments, the AUC_{inf} of Compound 1 is provided by administering about 100 mg of Compound 1 or a pharmaceutically acceptable salt thereof once a day.

In some embodiments, the present methods provide steady state plasma Cmax levels (or mean steady state plasma Cmax levels) of Compound 1 that correlate to one or more statistically significant therapeutic effects. In certain embodiments, the therapeutically effective steady state plasma Cmax levels (or mean steady state plasma Cmax levels) of Compound 1 provided by the methods of the present disclosure range from about 5 ng/ml to about 500 ng/mL, including about 5 ng/mL, 10 ng/mL, 20 ng/mL, 30 ng/ml, 40 ng/ml, 50 ng/mL, 60 ng/mL, about 70 ng/mL, about 80 ng/mL, about 90 ng/mL, about 100 ng/ml, about 110 ng/mL, about 120 ng/mL, about 130 ng/mL, about 140 ng/ml, about 150 ng/mL, about 160 ng/mL, about 170 ng/mL about 180 ng/mL, about 190 ng/ml, about 200 ng/ml, about 210 ng/mL, about 220 ng/mL, about 230 ng/mL, about 240 ng/mL, about 250 ng/ml, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, about 400 ng/mL, about 410 ng/mL, about 420 ng/mL, about 430 ng/mL, about 440 ng/mL, about 150 ng/mL, about 460 ng/mL, about 470 ng/ml about 480 ng/mL, about 490 ng/mL, about 500 ng/mL, about 510 ng/mL about 520 ng/mL, about 530 ng/mL, about 540 ng/mL, about 550 ng/mL, about 560 ng/mL, about 570 ng/mL about 580 ng/mL, about 590 ng/mL and about 600 ng/ml, including all ranges there between. In some embodiments, the therapeutically effective steady state plasma Cmax levels (or mean steady state plasma Cmax levels) of Compound 1 provided by the methods of the present disclosure are from about 100 ng/mL to about 275 ng/mL, including about 110 ng/mL, about 120 ng/mL, about 130 ng/mL, about 140 ng/ml, about 150 ng/mL, about 160 ng/mL, about 170 ng/ml about 180 ng/ml, about 190 ng/ml, about 200 ng/ml, about 210 ng/mL, about 220 ng/mL, about 230 ng/mL, about 240 ng/mL, about 250 ng/ml, about 260 ng/mL, about 270 ng/mL, including all ranges there between. In some embodiments, the therapeutically effective steady state plasma Cmax levels (or mean steady state plasma Cmax levels) of Compound 1 provided by the methods of the present disclosure are from about 125 ng/ml to about 250 ng/mL.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 15 mg. In further embodiments, the therapeutically effective steady state plasma Cmax levels (or mean steady state plasma Cmax levels) of Compound 1 is provided by administering about 15 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 20 mg. In further embodiments, the therapeutically effective steady state plasma Cmax levels (or mean steady state plasma Cmax levels) of Compound 1 is provided by administering about 10 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 20 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 25 mg. In further embodiments, the therapeutically effective steady state plasma Cmax levels (or mean steady state plasma Cmax levels) of Compound 1 is provided by administering about 25 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 30 mg. In further embodiments, the therapeutically effective steady state plasma Cmax levels (or mean steady state plasma Cmax levels) of Compound 1 is provided by administering about 15 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 30 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 35 mg. In further embodiments, the therapeutically effective steady state plasma Cmax levels (or mean steady state plasma Cmax levels) of Compound 1 is provided by administering about 35 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 40 mg. In further embodiments, the therapeutically effective steady state plasma Cmax levels (or mean steady state plasma Cmax levels) of Compound 1 is provided by administering about 20 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 40 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 45 mg. In further embodiments, the therapeutically effective steady state plasma Cmax levels (or mean steady state plasma Cmax levels) of Compound 1 is provided by administering about 45 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 50 mg. In further embodiments, the therapeutically effective steady state plasma Cmax levels (or mean steady state plasma Cmax levels) of Compound 1 is provided by administering about 25 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 50 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 55 mg. In further embodiments, the therapeutically effective steady state plasma Cmax levels (or mean steady state plasma Cmax levels) of Compound 1 is provided by administering about 55 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 60 mg. In further embodiments, the therapeutically effective steady state plasma Cmax levels (or mean steady state plasma Cmax levels) of Compound 1 is provided by administering about 30 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 60 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 65 mg. In further embodiments, the therapeutically effective steady state plasma Cmax levels (or mean steady state plasma Cmax levels) of Compound 1 is provided by administering about 65 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 70 mg. In further embodiments, the therapeutically effective steady state plasma Cmax levels (or mean steady state plasma Cmax levels) of Compound 1 is provided by administering about 35 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 70 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 75 mg. In further embodiments, the therapeutically effective steady state plasma Cmax levels (or mean steady state plasma Cmax levels) of Compound 1 is provided by administering about 75 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 80 mg. In further embodiments, the therapeutically effective steady state plasma Cmax levels (or mean steady state plasma Cmax levels) of Compound 1 is provided by administering about 40 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 80 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 85 mg. In further embodiments, the therapeutically effective steady state plasma Cmax levels (or mean steady state plasma Cmax levels) of Compound 1 is provided by administering about 85 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 90 mg. In further embodiments, the therapeutically effective steady state plasma Cmax levels (or mean steady state plasma Cmax levels) of Compound 1 is provided by administering about 45 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 90 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 95 mg. In further embodiments, the therapeutically effective steady state plasma Cmax levels (or mean steady state plasma Cmax levels) of Compound 1 is provided by administering about 95 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 100 mg. In further embodiments, the therapeutically effective steady state plasma Cmax levels (or mean steady state plasma Cmax levels) of Compound 1 is provided by administering about 50 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 100 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 105 mg. In further embodiments, the therapeutically effective steady state plasma Cmax levels (or mean steady state plasma Cmax levels) of Compound 1 is provided by administering about 105 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 110 mg. In further embodiments, the therapeutically effective steady state plasma Cmax levels (or mean steady state plasma Cmax levels) of Compound 1 is provided by administering about 55 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 110 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 115 mg. In further embodiments, the therapeutically effective steady state plasma Cmax levels (or mean steady state plasma Cmax levels) of Compound 1 is provided by administering about 115 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 120 mg. In further embodiments, the therapeutically effective steady state plasma Cmax levels (or mean steady state plasma Cmax levels) of Compound 1 is provided by administering about 60 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 120 mg of Compound 1 or a pharmaceutically acceptable salt thereof once a day.

In some embodiments, the present methods provide steady state plasma Cmax levels (or mean steady state plasma Cmax levels) of Compound 1 that do not exceed 500 ng/ml. In certain embodiments, the therapeutically effective steady state plasma Cmax levels of Compound 1 provided by the methods of the present disclosure do not exceed about 500 ng/ml, including less than about 500 ng/mL, less than about 475 ng/mL, less than about 450 ng/ml, than about 425 ng/ml, less than about 400 ng/ml, less than about 375 ng/ml, than about 350 ng/mL, less than about 325 ng/mL, and less than about 300 ng/mL.

In some embodiments, administration of Compound 1 in the evening provides steady state plasma Cmax levels (or mean steady state plasma Cmax levels) of Compound 1 that are reduced as compared to administration of Compound 1 in the morning. In some embodiments, Cmax after evening dosing is reduced by at least about 25%, for example, about 25%, about 30%, about 35%, about 40%, about 45%, about 50%, about 55%, about 60%, about 65%, about 70%, or about 75% compared to Cmax after morning dosing. In some embodiments, Cmax after evening dosing is less than about 327 ng/mL, for example, about 327 ng/ml, about 300 ng/mL, about 275 ng/mL, about 250 ng/ml, about 225 ng/mL, about 200 ng/ml, about 175 ng/mL, or about 150 ng/mL.

In some embodiments, administration of Compound 1 in the evening provides a tₘₐₓ of Compound 1 that are increased as compared to administration of Compound 1 in the morning. In some embodiments, tₘₐₓ after evening dosing is increased by at least about 50%, for example, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% compared to tₘₐₓ after morning dosing. In some embodiments, Cmax after evening dosing is greater than about 1 hour, for example, about 1.25 hours, about 1.5 hours, about 1.75 hours, about 2 hours, about 2.25 hours, about 2.5 hours, about 2.75 hours, about 3 hours, about 3.5 hours, about 3.75 hours, about 4 hours, or more.

In some embodiments, administration of Compound 1 or a pharmaceutically acceptable salt thereof in the evening provides an AUC_{inf} that is substantially similar to the AUC_{inf} provided by administration of Compound 1 or a pharmaceutically acceptable salt thereof in the morning. In some embodiments, an AUC_{inf} provided by evening dosing that is substantially similar to an AUC_{inf} provided by morning dosing is within 15% of the AUC_{inf} provided by morning dosing, for example, within about 1%, about 2%, about 3%, about 4%, about 5%, about 6%, about 7%, about 8%, about 9%, about 10%, about 11%, about 12%, about 13%, about 14%, or about 15% of the AUC_{inf} provided by morning dosing. For example, an AUC_{inf} of 2510 ng*hr/mL provided by evening dosing is substantially similar to an AUC_{inf} of 2610 ng*hr/mL provided by morning dosing. In some embodiments, an AUC_{inf} provided by evening dosing that is substantially similar to an AUC_{inf} provided by morning dosing is the same as the AUC_{inf} provided by morning dosing.

In some embodiments of the present methods, reducing the symptoms of a neurological disorder, such as epilepsy or an epilepsy syndrome (e.g., seizures) comprises administration of Compound 1 or a pharmaceutically acceptable salt thereof to a subject in need thereof.

In some embodiments, administration of Compound 1 or a pharmaceutically acceptable salt thereof to a subject in need thereof provides an increase in qEEG beta frequency band compared to baseline (i.e., prior to administering Compound 1 or a pharmaceutically acceptable salt thereof). In some embodiments, the increase in qEEG beta frequency band is from about 1.1-times to about 4-times compared to baseline (i.e., prior to administering Compound 1 or a pharmaceutically acceptable salt thereof). In some embodiments, the administration of Compound 1 or a pharmaceutically acceptable salt thereof provides a plasma concentration from about 1 ng/mL to about 500 ng/mL and an about 1.1-times to about 4-times increase in qEEG beta frequency band compared to baseline. In some embodiments, the increase in qEEG beta frequency band occurs 0.5 to 5 hours after dosing (e.g., about 0.5, 1, 1.5, 2, 2.5, 3, 3.5, 4, 4.5, or 5 hours after dosing). In some embodiments, the increase in beta frequency band is associated with a reduction of side effects selected from somnolence, sedation, dizziness, lightheadedness, fatigue, euphoria, feeling drunk, feeling abnormal, headache, cognitive disorder, disturbance of attention, memory impairment, slowed cognition, decreased alertness, constipation, and diarrhea. In some embodiments, administration of Compound 1 or a pharmaceutically acceptable salt thereof is nightly administration.

In some embodiments, administration of Compound 1 or a pharmaceutically acceptable salt thereof to a subject in need thereof provides an increase in qEEG alpha frequency band compared to baseline (i.e., prior to administering Compound 1 or a pharmaceutically acceptable salt thereof). In some embodiments, the increase in qEEG alpha frequency band is from about 1.1-times to about 4-times compared to baseline (i.e., prior to administering Compound 1 or a pharmaceutically acceptable salt thereof). In some embodiments, the administration of Compound 1 or a pharmaceutically acceptable salt thereof provides a plasma concentration from about 1 ng/mL to about 500 ng/ml and an about 1.1-times to about 4-times increase in qEEG alpha frequency band compared to baseline. In some embodiments, the increase in qEEG alpha frequency band occurs 0.5 to 5 h after dosing (e.g., about 0.5, 1, 1.5, 2, 2.5, 3, 3.5, 4, 4.5, or 5 hours after dosing). In some embodiments, the increase in alpha frequency band is associated with a reduction of side effects selected from the group consisting of somnolence, sedation, dizziness, lightheadedness, fatigue, euphoria, feeling drunk, feeling abnormal, headache, cognitive disorder, disturbance of attention, memory impairment, slowed cognition, decreased alertness, constipation, and diarrhea. In some embodiments, administration of Compound 1 or a pharmaceutically acceptable salt thereof is nightly administration.

In some embodiments, administration of Compound 1 or a pharmaceutically acceptable salt thereof to a subject in need thereof provides an increase in qEEG alpha frequency band and/or beta frequency band compared to baseline (i.e., prior to administering Compound 1 or a pharmaceutically acceptable salt thereof). In some embodiments, the increase in qEEG alpha frequency band and/or beta frequency band is from about 1.1-times to about 4-times compared to baseline (i.e., prior to administering Compound 1 or a pharmaceutically acceptable salt thereof). In some embodiments, the administration of Compound 1 or a pharmaceutically acceptable salt thereof provides a plasma concentration from about 1 ng/ml to about 500 mg/mL and an about 1.1-times to about 4-times increase in qEEG alpha frequency band and/or beta frequency band compared to baseline. In some embodiments, the increase in qEEG alpha frequency band and/or beta frequency band occurs 0.5 to 5 h after dosing (e.g., about 0.5, 1, 1.5, 2, 2.5, 3, 3.5, 4, 4.5, or 5 hours after dosing). In some embodiments, the increase in alpha frequency band and/or beta frequency band is associated with a reduction of side effects selected from the group consisting of somnolence, sedation, dizziness, lightheadedness, fatigue, euphoria, feeling drunk, feeling abnormal, headache, cognitive disorder, disturbance of attention, memory impairment, slowed cognition, decreased alertness, constipation, and diarrhea. In some embodiments, administration of Compound 1 or a pharmaceutically acceptable salt thereof is nightly administration.

In some embodiments, the subject in need thereof is a patient that demonstrates a response in the reduction of symptoms related to a neurological disorder, such as epilepsy or an epilepsy syndrome.

### EXAMPLES

The present disclosure is further illustrated by reference to the following Examples. However, it is noted that the Examples, like the embodiments described above, are illustrative and are not to be construed as restricting the scope of the disclosure in any way.

### Example 1: Compound 1 Protects Against Phenylenetetrazole-Evoked Seizures

Phenylenetetrazole (PTZ) is a GABA receptor antagonist that may be used to create a chemically-induced seizure model in mice. Accordingly, the effect on PTZ-evoked seizures was evaluated for vehicle controls, valproate (VPA), and varying dosages of Compound 1. Six-week old CD-1 male mice (25-35 g) were used in the following experiment. Study groups (n=12 for each group) were as follows:
(1) Saline (Vehicle for VPA), 10 ml/kg, i.p.);
(2) VPA (400 mg/kg, 10 ml/kg, i.p);
(3) 15% Captisol® (Vehicle for Compound 1, 10 ml/kg, p.o.);
(4) Compound 1 (3 mg/kg, p.o.);
(5) Compound 1 (10 mg/kg, p.o); and
(6) Compound 1 (30 mg/kg, p.o.).

Dosing solutions were made from stock solutions of the highest dose and diluted down for the lower doses. The compound is expected to be in a clear solution at 3 mg/ml in 15% Captisol. A long sonication may be performed to obtain a clear solution. All solutions were protected from light and made fresh daily for two experiment days. Mice were brought to the test room at least one hour before the PTZ test. Each dose was administered 30 minutes prior to administration of PTZ (120 mg/kg s.c., 10 ml/kg).

Sedative side effects were observed at the time right before dosing PTZ of each mouse, and marked with the following 4-grade scale: 1) None: normal behavior; 2) Mild sedation: mouse showed less locomotion or immobility when stayed alone, but showed normal locomotion activity if it was touched by hand; 3) Moderate sedation: mouse showed immobility when stayed alone and reduced capability of locomotion when it was pushed or touched by hand; and 4) Severe sedation: mouse completely lost the capability of movement.

Latency and total numbers of clonic and tonic seizures for 30 minutes were recorded, as well as latency to death and percent death, with a cut-off time of 30 minutes. The latency and number of seizures for each mouse was recorded by a person blinded to the treatment. For experimental endpoints, a clonic seizure was categorized as a clonus of whole body lasting for over 3 seconds, with an accompanying loss of righting reflex. A tonic seizure was categorized as a rigid extension of four limbs that exceeds a 90°-angle with the body.

Data Analysis: Two-tailed Student's t-test was used to detect significant differences in latency to clonic and tonic seizure and latency to death between the vehicle 1 (saline) and VPA groups. Mann-Whitney test was used to detect significant differences in the number of clonic or tonic seizure between vehicle 1 (saline) and VPA groups. One-way analysis of variance followed by Dunnett's test was used to detect significant differences in latency to clonic and tonic seizure and latency to death between vehicle 2 (Captisol®) and the Compound 1 groups. Kruskal-Wallis followed by Dunn's test was used to detect significant differences in the number of clonic or tonic seizure between vehicle 2 (Captisol®) and the Compound 1 groups. All statistical analysis are conducted using GraphPadPrism 7.0.

Terminal plasma and brain tissue samples were collected. Mice were anesthetized with CO₂. 500 ul whole blood was collected via cardiac puncture in a tube with 10 ul-EDTAK2 (blood: 15% EDTAK2=100 ul: 2 ul), placed in wet ice, and centrifuged at 2000 g, 5 min at 4° C., within 10 minutes.

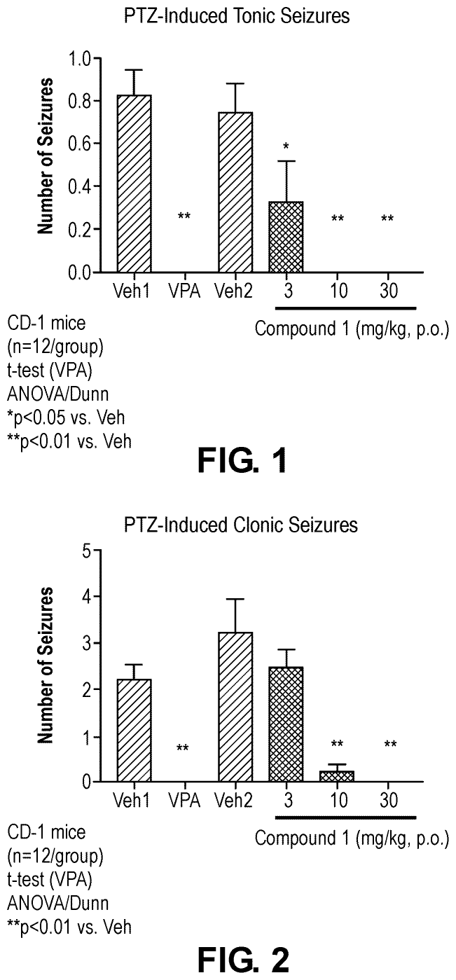

As shown in FIG. 1, administration of 3 mg/kg of Compound 1 significantly reduced (p<0.05) the number of tonic seizures as compared to a vehicle control, and administration of 10 mg/kg and 30 mg/kg of Compound 1 eliminated seizure activity (p<0.01 vs. vehicle control). Likewise, as shown in FIG. 2, administration of 10 mg/kg of Compound 1 significantly reduced (p<0.01) the number of clonic seizures as compared to a vehicle control, and administration of 30 mg/kg of Compound 1 eliminated seizure activity (p<0.01 vs. vehicle control).

### Example 2—Combination Treatment of Compound 1 with Compound A in a PTZ-Evoked Seizures

The combination of Compound 1 with Compound A for the treatment of seizures was investigated using a PTZ acute seizure model. Accordingly, the effect on PTZ-evoked seizures was evaluated for a vehicle control, Compound A alone, Compound 1 alone, and a combination of Compound 1 and Compound A.

Six-week old CD-1 male mice (25-35 g) were used in the following experiment. Study groups (n=12 for each group) were as follows:
(1) 35% 2-hydroxypropyl-beta-cyclodextrin (HPBCD), the vehicle for Compound 1 and Compound A (10 ml/kg, p.o.);
(2) Compound A (1 mg/kg, p.o.);
(3) Compound A (3 mg/kg, p.o.);
(4) Compound A (6 mg/kg, p.o.);
(5) Compound A (10 mg/kg, p.o);
(6) Compound 1 (10 mg/kg, p.o.); and
(7) Compound A (1 mg/kg, p.o.) and Compound 1 (10 mg/kg, p.o.).

In each instance, 30 minutes after administration of the vehicle or compound(s), PTZ was administered at 120 mg/kg, s.c., 10 ml/kg.

Dosing solutions were made from stock solutions of the highest dose and diluted down for the lower doses. All solutions were protected from light and made fresh daily for two experiment days. Mice were brought to the test room at least one hour before the PTZ test. Each mouse was dosed as indicated above, followed 30 minutes later by administration of PTZ (120 mg/kg s.c., 10) ml/kg).

Latency and total numbers of clonic/tonic seizures were recorded for 30 minutes, and latency to death was also recorded up to 30 minutes. A clonic seizure was defined as clonus of whole body lasting for over 3 seconds, with an accompanying loss of righting reflex. A tonic seizure was defined as a rigid extension of four limbs that exceeded a 90-degree angle with the body. The latency and number of seizures for each mouse was recorded by an individual blinded to the treatment. Sedative side effect was observed at the time right before dozing PTZ of each mouse, and marked in a 4-grade scale as follows: (1) none-normal behavior; (2) mild sedation-mouse showed less locomotion or immobility when stayed alone, but showed normal locomotion activity if touched by hand; (3) moderate sedation-mouse showed immobility when stayed alone and reduced capability of locomotion when pushed or touched by hand; and (4) severe sedation-mouse completely lost the capability of movement.

A two-tailed student's t-test was used to detect significant differences in latency to clonic and tonic seizure and latency to death between the vehicle and compound groups. Mann-Whitney test was used to detect significant differences in the number of clonic or tonic seizures between the vehicle and compound groups. One-way analysis of variance followed by Dunnett's test was used to detect significant differences in latency to clonic and tonic seizure and latency to death between vehicle and compound groups. Kruskal-Wallis followed by Dunn's test was used to detect significant differences in the number of clonic or tonic seizure between vehicle and compound groups. All statistical analysis were conducted using GraphPadPrism 8.0.

Terminal plasma and brain tissue samples were collected from mice immediately after completion of PTZ measurements for pharmacokinetic analysis. Mice were anesthetized with CO₂. 500 ul of whole blood was collected via cardiac puncture in a tube with 10 ul-EDTAK2 (blood: 15% EDTAK2=100 ul: 2 ul). Whole brain was collected, weighed, and placed on dry ice. The tube containing blood sample was placed in wet ice and centrifuged (2000 g, 5 minutes at 4° C.) within 10 minutes. Supernatant plasma samples were pipetted into blank EP tubes on dry ice.

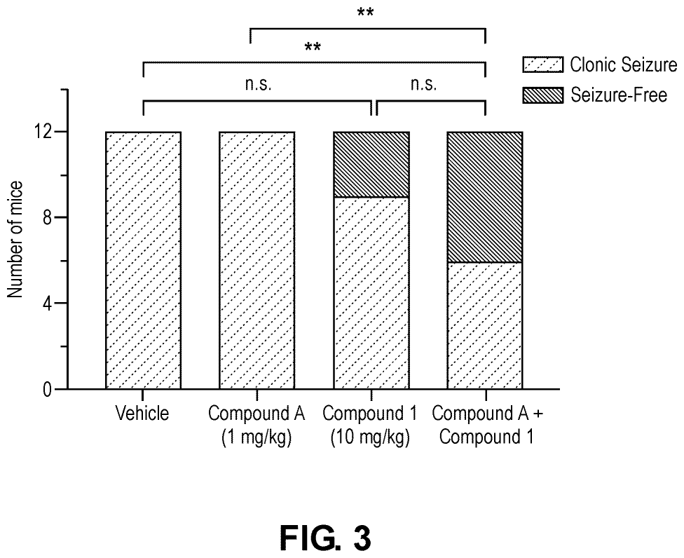

As shown in FIG. 3, administration of a combination of Compound 1 (10 mg/kg) and Compound A (1 mg/kg) tended to potentiate anticonvulsant effect as compared to administration of a vehicle control or either Compound A (1 mg/kg) alone or Compound 1 (10 mg/kg) alone. In FIG. 3, ** indicates p<0.01, by Fisher's exact test. In the group administered the combination of Compound A (1 mg/kg) and Compound 1 (10 mg/kg), six of the twelve mice were seizure-free, as compared to 3 of the twelve mice administered Compound 1 (10 mg/kg) alone (n.s.) and none of the twelve mice administered either Compound A (1 mg/kg) alone or vehicle (p<0.01).

### Prophetic Example 3—Combination Treatment of Compound 1 with a Sodium Channel Blocker

As disclosed herein, a GABA_{A} PAM such as Compound 1 may be combined with a sodium-channel blocker for use as an anticonvulsant, including for the treatment of epilepsy and epilepsy-related disorders.

As an initial step, data for Compound 1 and a sodium-channel blocker (e.g., Compound B) will be gathered individually in the PTZ-evoked seizure model and/or a 6-Hz acute seizure models. Isobolographic analysis will then be conducted to determine a fixed dose ratio of the sodium channel blocker to Compound 1, wherein the PTZ and/or the 6-Hz acute seizure models may be used. Next, dose-response studies with the selected sodium channel blocker. Compound 1 dose ratio will be conducted in target indication specific models, including a Cdkl5^{+/-} mouse model, a hippocampal kainate model, a Tuberous Sclerosis Complex (TSC) mouse model, and/or a Maximal Electric Shock (MES) model. In a 6-Hz acute seizure model or MES model, animals (e.g., mice) may be administered a dosage of vehicle or test compound(s) 30 minutes prior to electrical stimulus. Exemplary conditions may include transauricular (50 Hz, 0.8s, 10 ms square pulse width, 50 mA) and corneal electroshocks (6 Hz, 3s, 0.3 ms rectangular pulse width, 32 mA) for MES and 6-Hz experiments, respectively. Mice may then be observed for the presence or absence of full tonic hindlimb extension (e.g., for MES), or psychomotor seizures defined as stun/immobility, forelimb clonus, Straub tail, and later head movement (e.g., for 6-Hz).

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

1. A method of treating a neurological disorder in a patient in need thereof comprising orally administering a therapeutically effective amount of Compound 1: 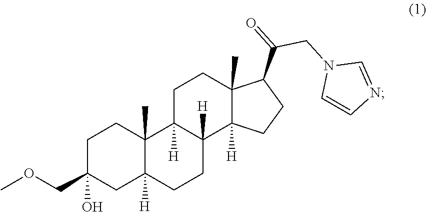
or a pharmaceutically acceptable salt thereof to a patient in need thereof.

2. The method of claim 1, wherein the neurological disorder is epilepsy or an epilepsy syndrome.

3. (canceled)

4. The method of claim 1, wherein the administration is about two hours after the evening meal or is about four hours after the evening meal.

5. (canceled)

6. The method of claim 1, wherein the administration is between about 5 p.m. and about 12 a.m.

7. The method of claim 1, wherein the evening is within about two hours prior to bedtime or at about bedtime.

8. (canceled)

9. The method according to claim 1, wherein about 5 mg to about 120 mg of Compound 1 or a pharmaceutically acceptable salt is administered per day.

10. The method according to claim 1, wherein about 40 mg of Compound 1 or a pharmaceutically acceptable salt thereof is administered per day.

11. The method according to claim 1, wherein about 60 mg of Compound 1 or a pharmaceutically acceptable salt thereof is administered per day.

12. The method according to claim 1, wherein said administering is for about 1 weeks to about 4 weeks.

13. The method according to claim 1, wherein after administering Compound 1 or a pharmaceutically acceptable salt thereof, the patient experiences a reduction of seizures as compared to prior to the administration of Compound 1 or a pharmaceutically acceptable salt thereof.

14. The method according to claim 13, wherein the seizures are clonic seizures or tonic seizures.

15. (canceled)

16. The method of claim 1, wherein administering Compound 1 or a pharmaceutically acceptable salt thereof provides a mean steady state AUC₀₋₂₄ of from about 600 ng·h/mL to about 900 ng·h/mL.

17. The method of claim 1, wherein administering Compound 1 or a pharmaceutically acceptable salt thereof provides a mean steady state Cmax of from about 125 ng/ml to about 250 ng/ml.

18. The method of claim 1, wherein the method further comprises administering at least one antiepileptic agent to the patient.

19. The method of claim 18, wherein the at least one antiepileptic agent is chosen from brivaracetam, cannabidiol, carbamazepine, clobazam, clonazepam, diazepam, divalproex, eslicarbazepine, ethosuximide, ezogabine, fenfluramine, felbamate, gabapentin, lacosamide, lamotrigine, levetiracetam, lorazepam, neuroactive steroids, oxcarbezepine, permpanel, phenobarbital, phenytoin, pregabalin, primidone, rufinamide, tigabine, topiramate, valproic acid, vigabatrin, and zonisamide.

20. The method of claim 1, wherein the Compound 1 is a citrate salt.

21. The method of claim 1, wherein the Compound 1 is a hemi-citrate salt.

22. The method of claim 1, wherein the method further comprises administering at least one sodium channel blocker to the patient.

23. The method of claim 22, wherein the at least one sodium channel blocker is Compound A: 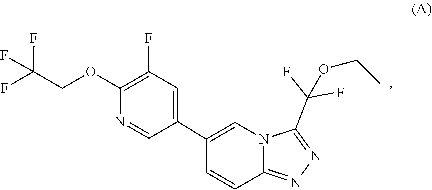 [SMILES: CCOC(F)(F)c1nnc2ccc(-c3cnc(OCC(F)(F)F)c(F)c3)cn12]
or a pharmaceutically acceptable salt thereof or Compound B 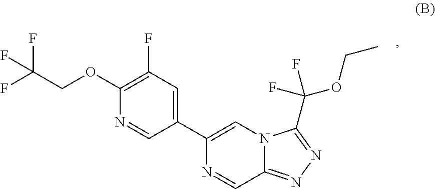 [SMILES: CCOC(F)(F)c1nnc2cnc(-c3cnc(OCC(F)(F)F)c(F)c3)cn12]
or a pharmaceutically acceptable salt thereof.

24. (canceled)

25. The method of claim 22, wherein about 0.1 mg to about 100 mg of the at least one sodium channel blocker is administered per day.
